(19) United States
(12) Patent Application Publication
(10) Pub. No.: US 2025/0304526 A1
(43) Pub. Date: Oct. 2, 2025

(54) HIGH AND MULTIPLE REDOX POTENTIAL, STABLE, AND SOLUBLE BIS-DIARYLAMINE DERIVATIVES AND USES THEREOF

(71) Applicant: University of Kentucky Research Foundation, Lexington, KY (US)

(72) Inventors: Chad Risko, Lexington, KY (US); Hussein Hijazi, Halifax (CA); Aman Kaur, Lexington, KY (US); Susan Odom, Lexington, KY (US)

(21) Appl. No.: 19/094,074

(22) Filed: Mar. 28, 2025

Related U.S. Application Data
(60) Provisional application No. 63/571,240, filed on Mar. 28, 2024.

(51) Int. Cl. C07C 211/56; C07C 211/54; C07C 217/92; H01M 8/02; H01M 8/18

(52) U.S. Cl. CPC C07C 211/56; C07C 211/54; C07C 217/92; H01M 8/02; H01M 8/188; H01M 2300/0025

(57) ABSTRACT

The present disclosure concerns the synthesis and investigation of a series of bis-triaryl amine compounds that span a range of multi-electron oxidation potentials. The compounds feature good solubility in acetonitrile and an exceptionally high oxidation potential compared to usual low oxidation potentials of such compounds. Radical cations of the compounds were synthesized and isolated as PF₆⁻ salts and characterized. Use of the compounds in a symmetrical RFB showed high cycling stability and capacity retention.

### CROSS-REFERENCE TO RELATED APPLICATIONS

The present disclosure claims priority to U.S. Provisional Patent Application 63/571,240, filed Mar. 28, 2024, the content of which is hereby incorporated by reference in its entirety.

### GOVERNMENT SUPPORT

The present disclosure was funded through grant 2019574 by the National Science Foundation. The Government may have certain rights to the invention.

### BACKGROUND

The growing demand for sustainable and renewable energy necessitates the development of new energy storage devices. In this regard, redox flow batteries (RFBs) stand out as promising option due to their attractive attributes including scalability, safety, and design flexibility. In RFB, energy is stored in the form of electrochemically active materials with disparate reduction potentials that are dissolved in electrolyte solutions in separate containers. During charging or discharging, the electrolyte solutions are pumped towards a power-converting reactor, in which they are oxidized or reduced to store or release energy before being returned to their respective containers. This design is particularly attractive since the energy capacity of such a battery (external containers size) is decoupled from its power capacity (reactor size), thereby allowing a cost-effective long-duration discharge. Nevertheless, the state-of-the-art energy storage active materials used in these devices suffer from low specific capacities as well as weak stabilities, leading to relatively inferior performances compared to other energy storage technologies such as lithium-ion batteries. Hence, there is a compelling need to derive new redox-active materials that can alleviate these problems. Organic redox-active molecules are of high interest as charge storing materials for RFB as they represent a promising alternative to the less abundant (e.g., vanadium), corrosive (e.g., bromine), and toxic (e.g., chromium) materials that are used in commercialized RFB. Moreover, non-aqueous organic media enable the operation of RFB at higher voltages due to the extended electrochemical stability window of organic solvents (e.g., up to 5 V in the case of acetonitrile) compared to their aqueous analogues (1.23 V), offering the possibility of improved energy densities.

Still, several issues must be overcome for non-aqueous organic RFB to be deployed for commercial applications. Specifically, the stability of redox-active material candidates in both their neutral and charged forms must be improved for long-term cycling stability. Additionally, solubilities of these materials in organic solvents must also increase to reach the energy densities and performance of other energy storage options.

To date, various classes of organic anolytes and catholytes have been studied and reported as potential RFB active materials, though none of them have demonstrated superior stability over popular vanadium-based redox-active materials, which are known to offer up to 20 years of operational lifetime.

### SUMMARY

A first aspect of the present disclosure, either alone or in combination with any other aspect herein, concerns an arylamine compound comprising a structure as set forth in Formula I and/or II:

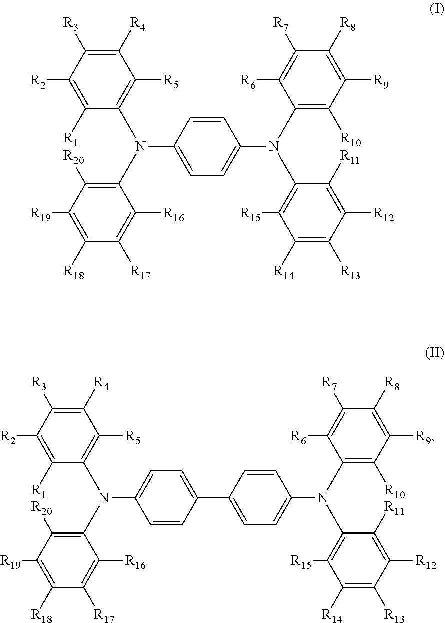

wherein: any of R₁ to R₂₀ are independently hydrogen, methoxy, trifluoromethyl, methyl, or diethylene glycol monomethyl ether. In some aspects, the central phenyl(s) may be appended with a further functional group, such as a methoxy group. In some aspects, two central phenyl rings of Formula II are separated by an intermediary alkyl chain, such as a methyl group.

A 2^{nd} aspect of the present disclosure, either alone or in combination with any other aspect herein, concerns the compound of the 1^{st} aspect, wherein all of R₁ top R₂₀ are hydrogen.

A 3^{rd} aspect of the present disclosure, either alone or in combination with any other aspect herein, concerns the compound of the 1^{st} aspect, wherein R₃, R₈, R₁₃, and R₁₈ are methoxy.

A 4^{th} aspect of the present disclosure, either alone or in combination with any other aspect herein, concerns the compound of the 3^{rd} aspect, wherein all further R groups are hydrogen.

A 5^{th} aspect of the present disclosure, either alone or in combination with any other aspect herein, concerns the compound of the 1^{st} aspect, wherein R₂, R₉, R₁₂, and R₁₉ are trifluoromethyl.

A 6^{th} aspect of the present disclosure, either alone or in combination with any other aspect herein, concerns the compound of the 5^{th} aspect, further wherein and R₃, R₈, R₁₃, and R₁₈ are methyl.

A 7^{th} aspect of the present disclosure, either alone or in combination with any other aspect herein, concerns the compound of the 1^{st} aspect, wherein R₂, R₉, R₁₂, and R₁₉ are trifluoromethyl.

An 8^{th} aspect of the present disclosure, either alone or in combination with any other aspect herein, concerns the compound of the 7^{th} aspect, further wherein R₅, R₆, R₁₅, and R₁₆ are methyl.

A 9^{th} aspect of the present disclosure, either alone or in combination with any other aspect herein, concerns the compound of the 1^{st} aspect, wherein R₃, R₈, R₁₃, and R₁₈ are trifluoromethyl.

A 10^{th} aspect of the present disclosure, either alone or in combination with any other aspect herein, concerns the compound of the 9^{th} aspect, further wherein R₅, R₆, R₁₅, and R₁₆ are methyl.

An 11^{th} aspect of the present disclosure, either alone or in combination with any other aspect herein, concerns the compound of the 1^{st} aspect, wherein R₄, R₉, R₁₄, and R₁₉ are trifluoromethyl.

A 12^{th} aspect of the present disclosure, either alone or in combination with any other aspect herein, concerns the compound of the 11^{th} aspect, further wherein R₃, R₈, R₁₃, and R₁₈ are diethylene glycol monomethyl ether.

A 13^{th} aspect of the present disclosure, either alone or in combination with any other aspect herein, concerns the compound of the 1^{st} aspect, wherein the compound is selected from the group consisting of:

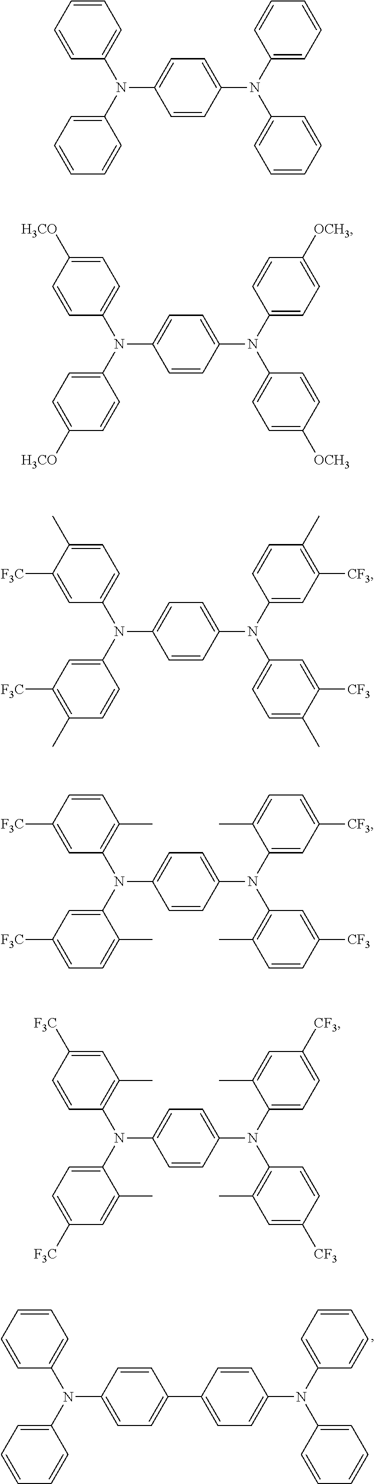 [SMILES: c1ccc(N(c2ccccc2)c2ccc(N(c3ccccc3)c3ccccc3)cc2)cc1 ; COc1ccc(N(c2ccc(OC)cc2)c2ccc(N(c3ccc(OC)cc3)c3ccc(OC)cc3)cc2)cc1 ; Cc1ccc(N(c2ccc(N(c3ccc(C)c(C(F)(F)F)c3)c3ccc(C)c(C(F)(F)F)c3)cc2)c2ccc(C)c(C(F)(F)F)c2)cc1C(F)(F)F ; Cc1ccc(C(F)(F)F)cc1N(c1ccc(N(c2cc(C(F)(F)F)ccc2C)c2cc(C(F)(F)F)ccc2C)cc1)c1cc(C(F)(F)F)ccc1C ; Cc1cc(C(F)(F)F)ccc1N(c1ccc(N(c2ccc(C(F)(F)F)cc2C)c2ccc(C(F)(F)F)cc2C)cc1)c1ccc(C(F)(F)F)cc1C ; c1ccc(N(c2ccccc2)c2ccc(-c3ccc(N(c4ccccc4)c4ccccc4)cc3)cc2)cc1]

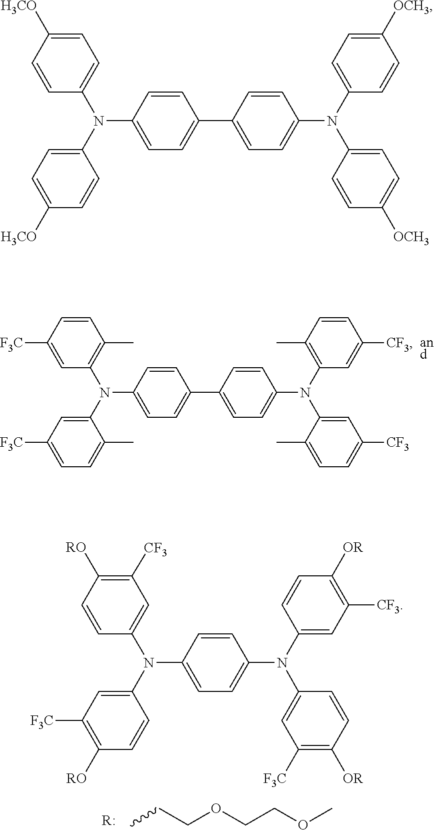

A 14^{th} aspect of the present disclosure, either alone or in combination with any other aspect herein, concerns the compound of the 1^{st} aspect, wherein the compound is

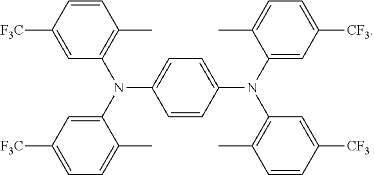 [SMILES: Cc1ccc(C(F)(F)F)cc1N(c1ccc(N(c2cc(C(F)(F)F)ccc2C)c2cc(C(F)(F)F)ccc2C)cc1)c1cc(C(F)(F)F)ccc1C]

A 15^{th} aspect of the present disclosure, either alone or in combination with any other aspect herein, concerns an electrolyte solution comprising the compound of claim 1^{st} aspect and a hexafluorophosphate salt.

A 16^{th} aspect of the present disclosure, either alone or in combination with any other aspect herein, concerns a redox flow battery comprising the electrolyte solution of the 15^{th} aspect. the compound of the 1^{st} aspect, wherein all of R₁ top R₂₀ are hydrogen.

A 17^{th} aspect of the present disclosure, either alone or in combination with any other aspect herein, concerns a redox-flow battery comprising the compound of the 1^{st} aspect.

An 18^{th} aspect of the present disclosure, either alone or in combination with any other aspect herein, concerns a method of providing electrical energy comprising flow of a cathoylte and an anolyte, wherein the catholyte comprises the compound of the 1^{st} aspect.

A 19^{th} aspect of the present disclosure, either alone or in combination with any other aspect herein, concerns the method of the 18^{th} aspect, wherein the catholyte further comprises a hexafluorophosphate salt.

### BRIEF DESCRIPTION OF THE DRAWINGS

FIG. 1 shows chemical structures of 1-4 synthesized and studied in this application.

FIG. 2 shows synthetic pathways for 1-4.

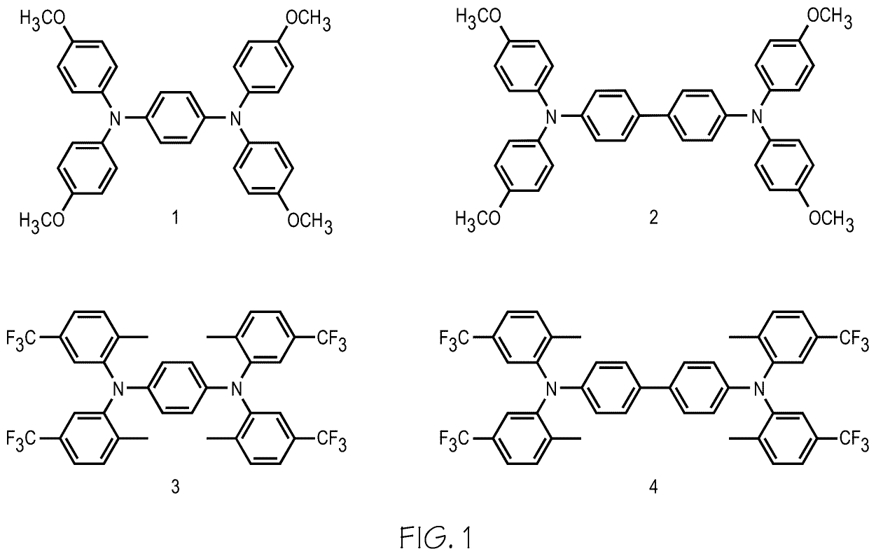

FIG. 1 shows UV-NIR absorption spectra showing the IVCT bands of 0.5 mM of 1 and 1^{+⋅}, 2 and 2^{+⋅}, 3 and 3^{+⋅} and 4 and 4^{+⋅} in pure CH₃CN. FIG. 3A shows 1 and 1⁺. FIG. 3B shows 2 and 2⁺. FIG. 3C shows 3 and 3⁺. FIG. 3D shows 4 and 4⁺.

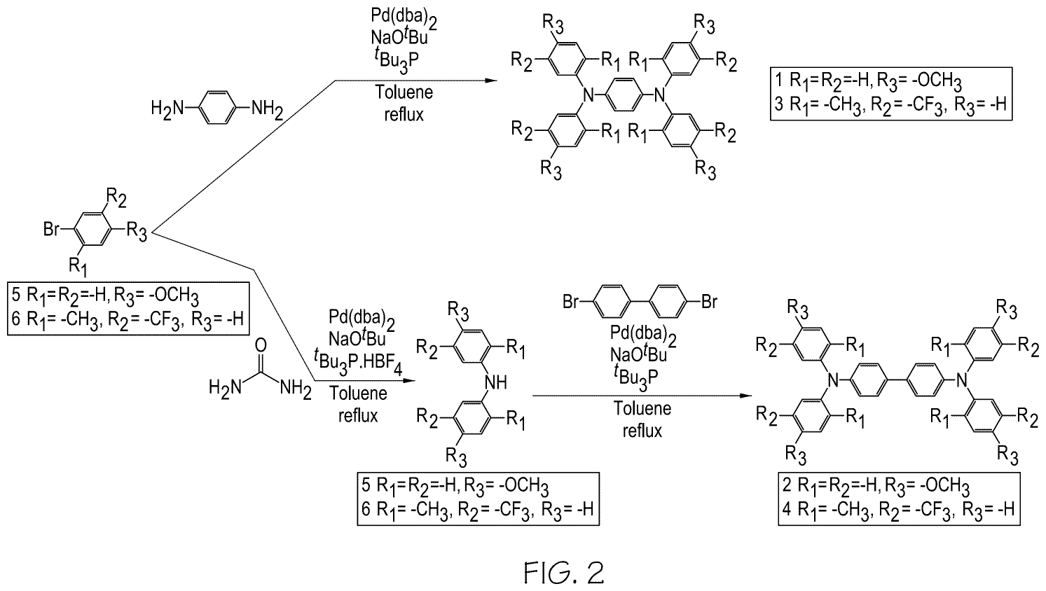

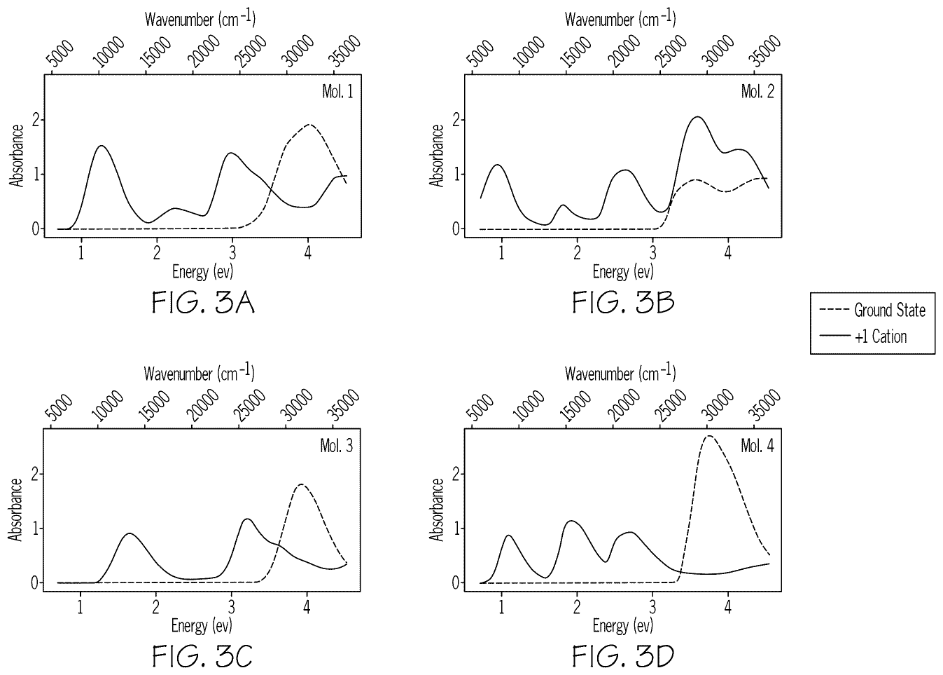

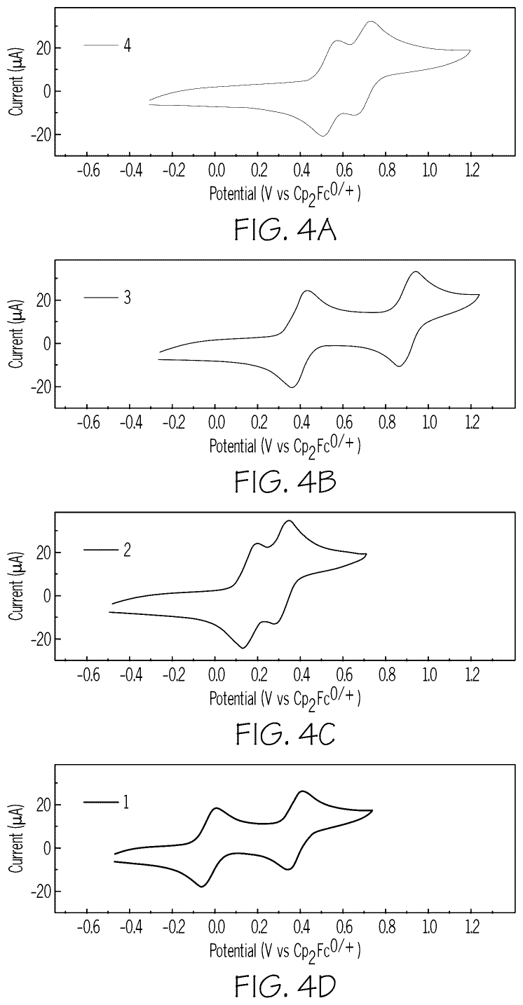

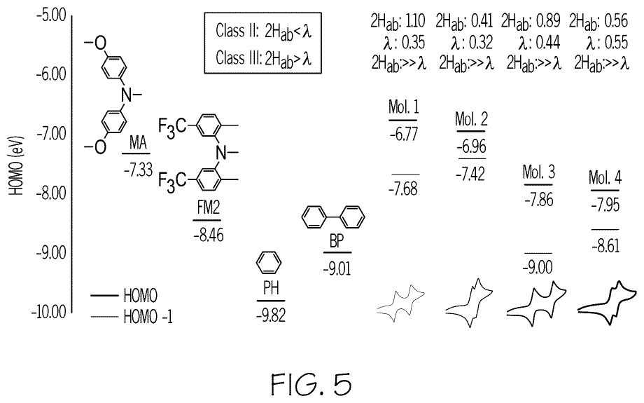

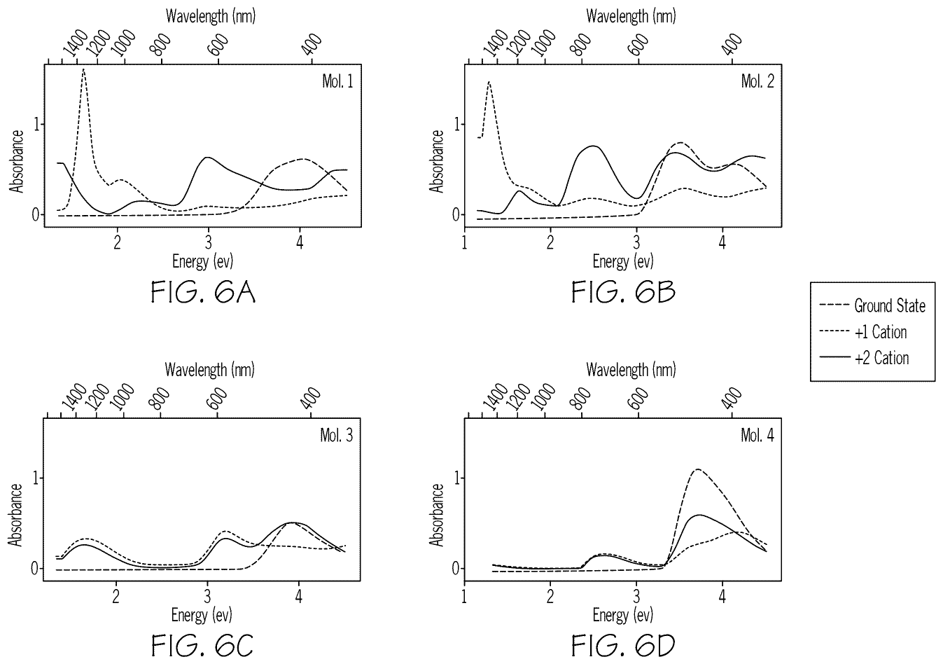

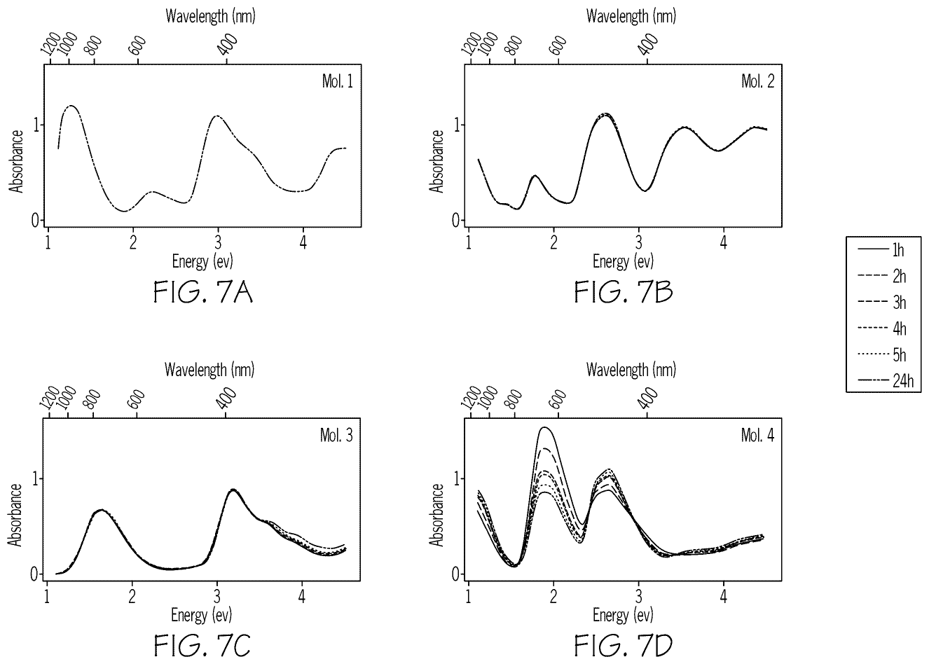

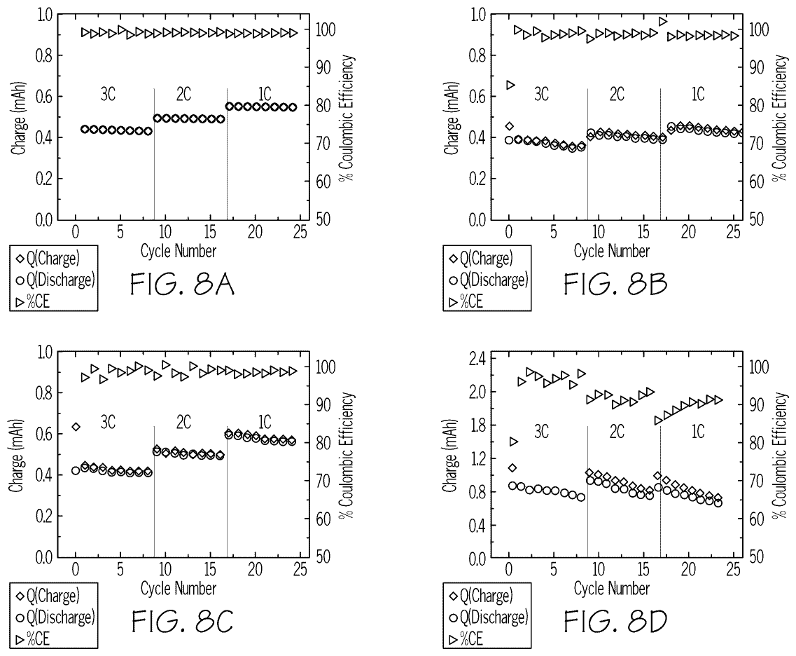

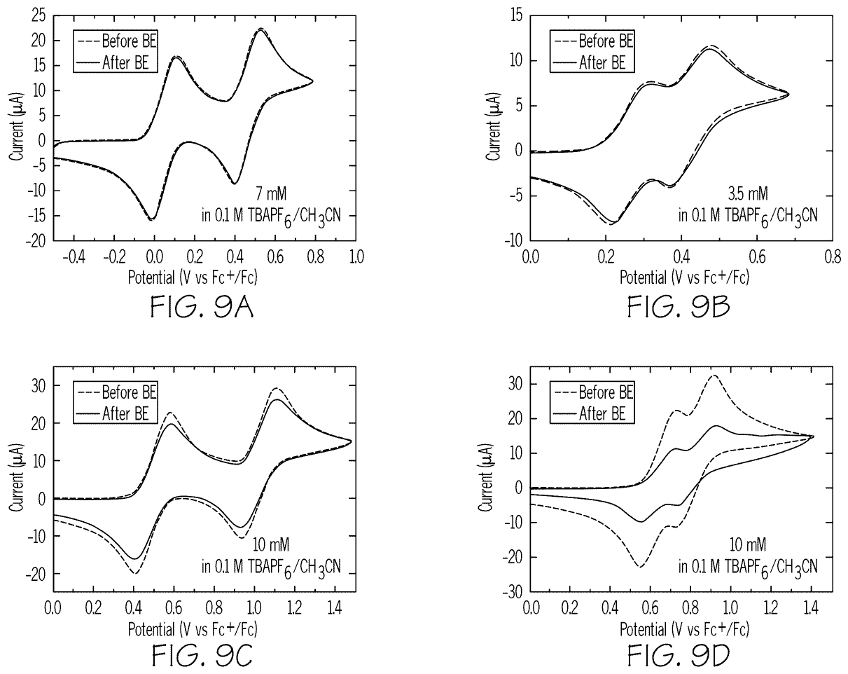

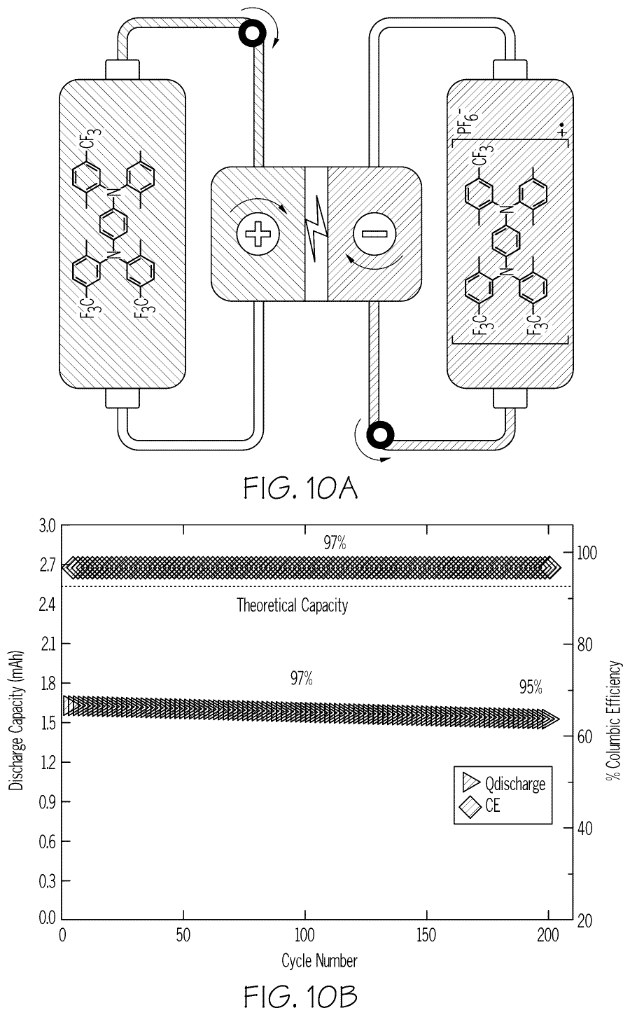

FIG. 2 shows cyclic voltammograms of 1 mM of 1, 2, 3, and 4 obtained in 0.2 M TBAPF₆/CH₃CN. Voltammograms were calibrated to Cp₂Fe^{0/+} at 0 V using ferrocene as an internal reference. Scan rate 0.1 V·s⁻¹.

FIG. 3 shows HOMO energies for the molecule components and the mixed-valence systems, performed with LC-ωHPBE/Def2SVP without omega-tuning so the energies could be compared. For the studied mixed-valence systems, both the HOMO and HOMO-1 are shown, and the electronic couplings (H_{ab}) and reorganization energies (λ) are specified. For the studied mixed-valence systems, the measured cyclic voltammogram is given above. All values are reported in eV.

FIG. 4 shows UV-vis absorption spectra of molecules 1-4 acquired at varying electrochemically generated oxidation states. Data is representative of n=3 trials. FIG. 6A shows molecule 1. FIG. 6B shows molecule 2. FIG. 6C shows molecule 3. FIG. 6D shows molecule 4.

FIG. 5 shows UV-vis absorption spectra of 0.5 mM of 1^{+⋅}, 2⁺, 3⁺, 4^{+⋅} (over time in 0.1 M TBAPF₆/CH₃CN, recorded at 0, 1, 2, 3, 4, 5 and 24 hours after dissolution. FIG. 7A shows 1⁺. FIG. 7B shows 2⁺. FIG. 7C shows 3⁺. FIG. 7D shows 4⁺.

FIG. 6 shows bulk electrolysis data of molecules 1-4 at 3C, 2C, and 1C charging rates. Both sides of the H-cell contain the active species at 7 mM for 1, 3.5 mM for 2 and 10 mM for 3 and 4 in 0.1 M TBAPF₆/CH₃CN. FIG. 8A shows molecule 1. FIG. 8B shows molecule 2. FIG. 8C shows molecule 3. FIG. 8D shows molecule 4.

FIG. 7 shows CVs measured in the 0.1M TBAPF₆/CH₃CN solutions of molecules 1 (1-4 before and after BE cycling. Scan rate is 0.2 V·s⁻¹. FIG. 9A shows molecule 1. FIG. 9B shows molecule 2. FIG. 9C shows molecule 3. FIG. 9D shows molecule 4.

FIG. 8 shows symmetric flow cell testing of compound 3 and its corresponding PF₆ salt at 10 mM in 0.1 M TBAPF₆/CH₃CN. FIG. 10A shows graphical depiction of the cell with chemical structures of compounds.

FIG. 10B shows discharge capacity (triangle) and coulombic efficiency (diamond) vs. cycle number (B). The theoretical capacity (dashed line) is 2.68 mAh.

### DESCRIPTION

Triaryl amines are a particularly promising class of organic molecules mainly due to their ability to form stable aminium radical cations. These molecules are ubiquitous, and they have been used as charge-transporting materials in dye-sensitized solar cells (DSSC) and organic light-emitting diodes (OLED), and even as polymer additives to prepare hybrid LiFePO₄(LFP) cathodes for lithium-ion batteries. Triarylamines have undergone little study as redox-active materials for RFB, perhaps as much due to their low oxidation potentials and low solubilities in polar organic solvents such as acetonitrile. The potential of triaryl amines as energy storage materials in RFB has not been widely explored. In the present disclosure, soluble triarylamine-based compounds are reported as catholytes for non-aqueous organic RFB; although soluble, the triarylamines exhibited relatively low oxidation potentials, which ultimately limits their eventual application.

Arylamines comprised of multiple π-conjugated nitrogen centers have been reported to exhibit better radical-cation stability upon oxidation as well as a wider range of electrochemical properties than simple triarylamines due to delocalization of the generated radical over the π-system. The bis-diarylamine derivatives provided herein offer high, multiple, and tunable redox potentials, solubility in organic solvents, and stability over redox cycling. The working examples demonstrate the synthesis and electrochemical and stability characterization along with use in a redux flow battery system. The compounds described herein provide redox-active molecules that can undergo multiple redox events over a wide redox potential while maintaining high degrees of solubility over long performance metrics.

In aspects, the arylamine compounds of the present description are of Formula I and/or II:

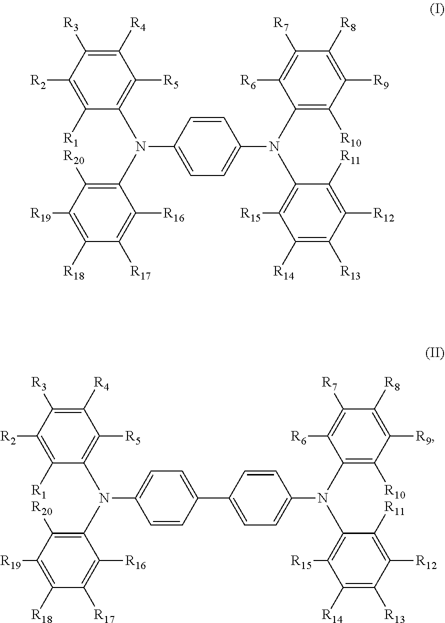

wherein: any of R₁ to R₂₀ are independently hydrogen, methoxy, trifluoromethyl, methyl, or diethylene glycol monomethyl ether. In some aspects, the central phenyl(s) may be appended with a further functional group, such as a methoxy group. In some aspects, two central phenyl rings of Formula II are separated by an intermediary alkyl chain, such as a methyl group.

In some aspects, the compound may include any one of the following:

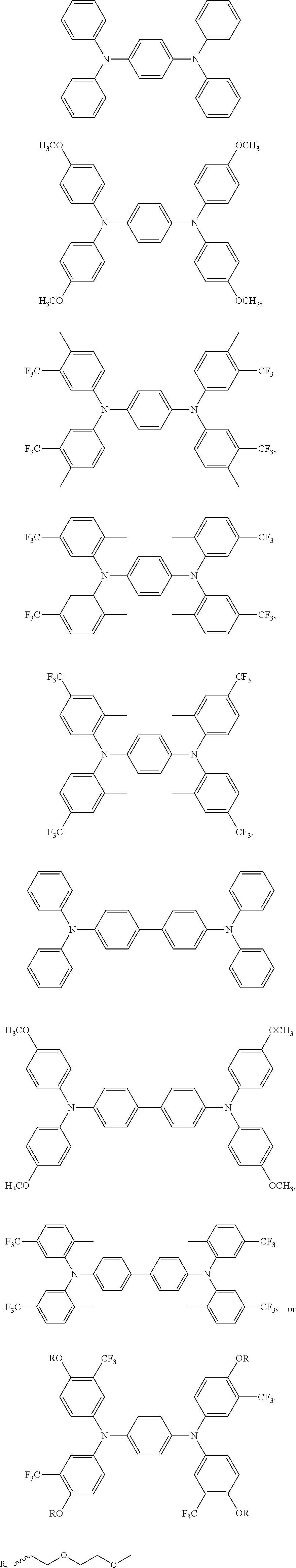

In aspects, R₃, R₈, R₁₃, and R₁₈ are methoxy. In further aspects, all remain R groups are hydrogen.

In aspects, all R groups, R₁-R₂₀, are hydrogen.

In aspects, R₂, R₉, R₁₂, and R₁₅ are trifluoromethyl or CF₃. In aspects, R₅, R₆, R₁₅, and R₁₆ are methyl or CH₃. In some aspects, R₂, R₉, R₁₂, and R₁₅ are trifluoromethyl or CF₃ and R₅, R₆, R₁₅, and R₁₆ are methyl or CH₃. In further aspects, all remaining R groups are hydrogen.

In aspects, R₂, R₉, R₁₂, and R₁₅ are trifluoromethyl or CF₃. In aspects, R₃, R₈, R₁₃, and R₁₈ are methyl or CH₃. In some aspects, R₂, R₉, R₁₂, and R₁₅ are trifluoromethyl or CF₃ and R₃, R₈, R₁₃, and R₁₈ are methyl or CH₃. are methyl or CH₃. In further aspects, all remaining R groups are hydrogen.

In aspects, R₄, R₉, R₁₃, and R₁₈ are —OR wherein R is diethylene glycol monomethyl ether. In some aspects, R₄, R₉, R₁₄, and R₁₉ are trifluoromethyl or CF₃. In some aspects, R₄, R₉, R₁₃, and R₁₈ are —OR wherein R is diethylene glycol monomethyl ether and R₄, R₉, R₁₄, and R₁₉ are trifluoromethyl or CF₃. In further aspects, all remaining R groups are hydrogen.

In aspects, R₃, R₈, R₁₃, and R₁₈ are trifluoromethyl or CF₃. In aspects, R₅, R₆, R₁₅, and R₁₆ are methyl or CH₃. In aspects, R₃, R₈, R₁₃, and R₁₈ are trifluoromethyl or CF₃ and R₅, R₆, R₁₅, and R₁₆ are methyl or CH₃. In further aspects, all remaining R groups are hydrogen.

In some aspects, the compounds of the present disclosure may be employed as part of an RFB. Arylamines comprised of multiple 7r-conjugated nitrogen centers provide a promising opportunity to open new avenues in non-aqueous organic RFB materials. As set forth in the examples, four modified bis-triarylamines (FIG. 1)bearing different functional groups and/or having different aryl bridges between the two nitrogen-based redox centers were synthesized and show how a molecular modification can dramatically increase the oxidation potentials of bis-triaryl amines and alter their solubilities in acetonitrile.

The bis-triarylamine derivatives 1-4 were synthesized in one or two steps starting from primary or secondary amine precursors (FIG. 1). Molecules 1 and 3 were synthesized via Buchwald-Hartwig coupling of 1,4-phenylenediamine and the suitable aryl bromide to afford products in good yields. For the synthesis of molecules 2 and 4, the corresponding diarylamines were first prepared by Buchwald-Hartwig coupling of the suitable aryl bromide derivatives with an excess of urea. The resulting intermediates were then coupled to 4,4′-dibromobiphenyl by Buchwald-Hartwig coupling to afford 2 and 4 (FIG. 2).

In some aspects, the present disclosure concerns fluorinated arylamines. As set forth herein, several novel fluorinated triarylamine derivatives were synthesized and studied as potential catholytes for RFB. These molecules exhibited improved solubilities in CH₃CN and higher oxidation potentials compared to their non-fluorinated derivatives. Radical cations were synthesized chemically and electrochemically and characterized by UV-vis-NIR where they feature IVCT bands, characteristic of mixed-valence compounds. BE and UV-vis spectroscopy experiments underline the good chemical and electrochemical stability of the neutral and charged forms of compound 3 in non-aqueous electrolyte systems. Finally, molecule 3 was employed in a symmetrical RFB showing high cycling stability. This class of molecules represents a powerful alternative for future applications in RFB. Molecular design and careful selection of functional groups play an essential role in improving the stability and solubility of these compounds.

The molecules disclosed here are related to those used as hole transport materials and/or emissive materials in organic light-emitting diodes and other organic semiconductor/organic electronics applications. It is notable that in these applications that the oxidation potentials are generally kept low so as to match the Fermi energies/work functions of contact electrodes. Further, the materials are often vacuum deposited, meaning the solubility is not an issue for consideration in the design.

In some aspects, the compounds disclosed herein can function as a catholyte in an RFB. For example, as set forth in the working examples herein, a symmetric flow cell was established to study the cycling stability of compound 3 and its corresponding hexafluorophosphate salt (FIG. 10A), initially used in both the compartments of the flow cell. Hence, the cell was assembled at 50% state-of-charge (SOC) and impedance spectrum was recorded prior to cycling using electrochemical impedance spectroscopy (EIS). The ohmic contribution (5.5 Ωcm² (Figure S)) estimated from the high frequency intercept of the Nyquist plot is consistent with other non-aqueous studies using Daramic separator. The capacity remained stable for 200 cycles, retaining 97% and 95% of first cycle capacity after 100 and 200 cycles respectively.

### EXAMPLES

### Materials

All materials were used as received. Bis(dibenzylideneacetone)palladium(0) and Palladium(II) acetate ≥98.0% were purchased from TCI chemicals and were stored and weighed in an argon-filled glovebox (MBraun, O₂<1 ppm, H₂O<0.5 ppm). 2-Bromo-1-methyl-4-(trifluoromethyl)benzene (99%) and Tri-t-butylphosphine (98%) were purchased from Oakwood Chemical. The latter was stored and weighed in an argon-filled glovebox. Sodium-tert-butanolate (NaO^{t}Bu)≥97% was purchased from Bean Town Chemical and was stored and weighed inside an argon-filled glovebox. O-Dianisidine ≥98.0% and N,N,N′,N′-Tetraphenyl-1,4-phenylenediamine ≥98% were purchased from TCI. 4-Bromoanisole 98% and 4,4′-Dibromobiphenyl 99% were purchased from Acros Organics. The silica gel (65×250 mesh) was purchased from Sorbent Technologies. Tetrabutylammonium hexafluorophosphate (TBAPF₆, >99%), p-Phenylenediamine and Urea were purchased from Sigma Aldrich. Anhydrous acetonitrile (CH₃CN, ≥99.9%) and anhydrous toluene (99.8%) were purchased from Alfa Aesar. ¹H and ¹³C NMR spectra were obtained on a 400 MHz Bruker Avance NEO (equipped with a Smart Probe) in DMSO-d₆ from Cambridge Isotope Laboratories. CV measurements and BE experiments were performed in a nitrogen-filled dry box.

### N,N,N′,N′-tetrakis(4-methoxyphenyl)-1,4-phenylenediamine

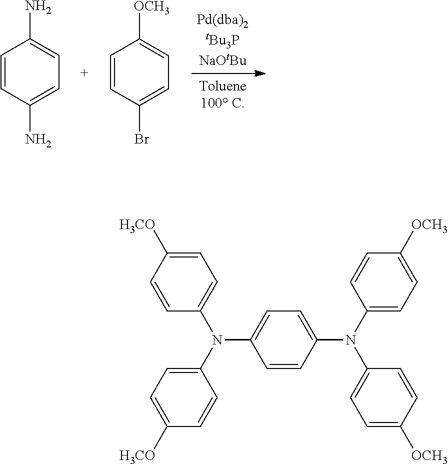 [SMILES: Nc1ccc(N)cc1 ; COc1ccc(Br)cc1 ; COc1ccc(N(c2ccc(OC)cc2)c2ccc(N(c3ccc(OC)cc3)c3ccc(OC)cc3)cc2)cc1]

Molecule 1: Synthesized according to published procedure.11 1,4-phenylenediamine (500 mg, 4.6 mmol, 1 eq.), 4-Bromo-anisol (3.8 g, 20.3 mmol, 4.4 eq.) and NaO^{t}Bu (2.7 g, 27.8 mmol, 6 eq.) were added to a dry round bottom flask containing 50 mL anhydrous toluene under nitrogen atmosphere. The mixture was degassed with a nitrogen stream for 15 min after which Pd(dba)₂ (53 mg, 0.09 mmol, 0.02 eq.) and ^{t}Bu₃P (15 mg, 0.074 mmol, 0.016 eq.) dissolved in 150 mL anhydrous toluene were added and nitrogen purging continued for 10 min. Then, the reaction mixture was heated to reflux and kept stirring overnight under nitrogen atmosphere. After that the reaction was allowed to cool down to RT, diluted with EtOAc and extracted from water by EtOAc. The organic layer was dried over MgSO₄, filtered and poured into MeOH. The solid precipitate was collected by filtration to afford a beige powder as product with a yield of 50%.

¹H NMR (400 MHz, DMSO-d6): δ=6.91 (m, 8H), 6.84 (m, 8H), 6.73 (s, 4H), 3.7 (s, 12H). ¹³C NMR (100 MHz, DMSO): 154.96, 142.16, 140.86, 125.22, 122.66, 114.79, 55.19.

### N,N,N′,N′-tetrakis(4-methoxyphenyl)-1,1′-biphenyl-4,4′-diamine

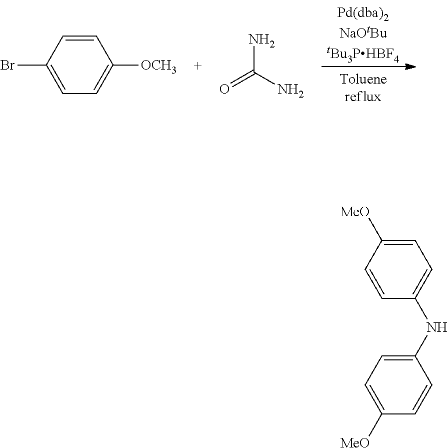

Molecule 2: In 250 mL dry round bottom flask, the bromo anisol compound (12.4 g, 66 mmol, 2 eq.) urea (2 g, 33 mmol, 1 eq.), NaO^{t}Bu (20 g, 208 mmol, 6 eq.) and ^{t}Bu₃·HBF₄ (580 mg, 2 mmol, 0.06 eq.) were dissolved in anhydrous toluene. The reaction mixture was degassed with nitrogen stream for 15 min and then Pd(dba)₂ (379 mg, 0.66 mmol, 0.06 eq.) was added. The reaction was then heated to 100° C. and kept stirring for 12 hours under a nitrogen atmosphere. The reaction was extracted by EtOAc from water. The organic layer was dried over Na₂SO₄ and concentrated under reduced pressure. The crude product was purified with silica gel column chromatography using hexanes as eluent to afford the pure secondary amine product as yellow flakes.

¹H NMR (400 MHz, CDCl₃): δ=6.94 (m, 4H), 6.85 (m, 4H), 5.33 (s, broad, 1H), 3.79 (s, 6H). ¹³C NMR (100 MHz, CDCl₃): δ=154.32, 138.07, 119.63, 114.81, 55.73.

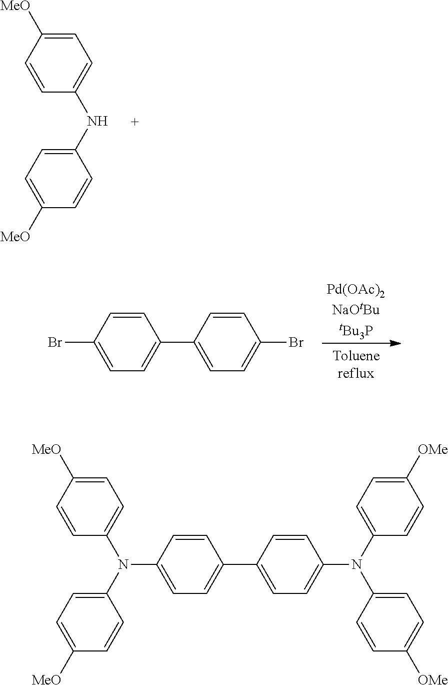 [SMILES: COc1ccc(Nc2ccc(OC)cc2)cc1 ; Brc1ccc(-c2ccc(Br)cc2)cc1 ; COc1ccc(N(c2ccc(OC)cc2)c2ccc(-c3ccc(N(c4ccc(OC)cc4)c4ccc(OC)cc4)cc3)cc2)cc1]

In a 50 mL dry round bottom flask equipped with a magnetic stirrer, was added 20 mL of anhydrous toluene. The secondary compound (3.8 g, 17 mmol, 2.1 eq.) and 4,4′-biphenyl (2.5 g, 8 mmol, 1 eq.) and NaO^{t}Bu (1.92 g, 20 mmol, 2.5 eq.) were added under nitrogen atmosphere. The mixture was then degassed with nitrogen for 30 min. After that, Pd(OAc)₂ (72 mg, 0.32 mmol, 0.04 eq.) and ^{t}Bu₃P (52 mg, 0.26 mmol, 0.032 eq.) dissolved in 5 mL anhydrous toluene were added. The mixture was refluxed overnight under a nitrogen atmosphere. The reaction mixture was then cooled, and product was precipitated by adding methanol. The product was filtered and recrystallized from EtOAc to provide yellow crystals with a yield of 60%.

¹H NMR (400 MHz, CDCl₃): δ=7.38-7.35 (d, J=8.61 Hz, 4H), 7.09-7.07 (d, J=8.89 Hz, 8H), 6.98-6.97 (d, J=8.61 Hz, 1.94, 4H), 6.85-6.83 (d, J=8.89 Hz, 8H), 3.8 (s, 12H). ¹³C NMR (100 MHz, CDCl₃): 155.84, 147.57, 141.21, 133.18, 127.03, 126.53, 121.22, 114.79, 55.63.

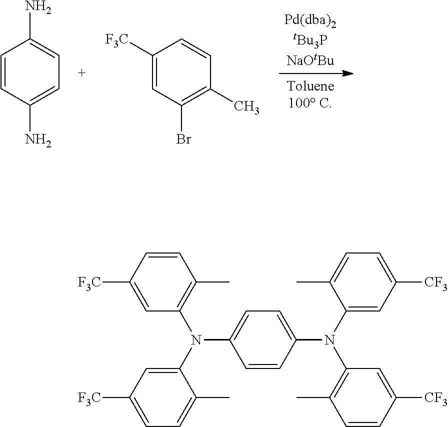 [SMILES: Nc1ccc(N)cc1 ; Cc1ccc(C(F)(F)F)cc1Br ; Cc1ccc(C(F)(F)F)cc1N(c1ccc(N(c2cc(C(F)(F)F)ccc2C)c2cc(C(F)(F)F)ccc2C)cc1)c1cc(C(F)(F)F)ccc1C]

For molecule 3, 1,4-phenylenediamine (760 mg, 7 mmol, 1 eq.), 2-Bromo-1-methyl-4-(trifluoromethyl)benzene (6.7 g, 28 mmol, 4 eq.) and NaO^{t}Bu (5.4 g, 56 mmol, 8 eq.) were added to a dry Schlenk flask containing 150 mL anhydrous toluene under nitrogen atmosphere. The mixture was degassed with a nitrogen stream for 15 min after which Pd(dba)₂ (242 mg, 0.4 mmol, 0.06 eq.) and ^{I}Bu₃P (327 mg, 1.6 mmol, 0.23 eq.) dissolved in 5 mL anhydrous toluene were added and nitrogen purging continued for 10 min. Then, the reaction mixture was heated to reflux and kept stirring overnight under nitrogen atmosphere. After that the reaction was allowed to cool down to RT, diluted with EtOAc and extracted twice with cold ice water. The combined organic layers were dried over Na₂SO₄ and the solvent was removed under reduced pressure. The crude product purified by silica gel column chromatography using hexanes as eluent. The product was then collected and concentrated via reduced pressure with a yield 90% (m=4.63 g) of white powder.

¹H NMR (400 MHz, CDCl₃): δ=7.31 (m, 8H), 7.11 (s, 4H), 6.64 (s, 4H), 2.03 (s, 12H). ¹³C NMR (100 MHz, CDCl₃): δ=146.44, 142.86, 138, 132.69, 129.84 (q, J=32.5 Hz), 125.39, 123.32, 123.04, 122.68, 121.46, 19.19. ¹⁹F-NMR (376.5 MHz, CD₃CN): δ=−62.47 (s)

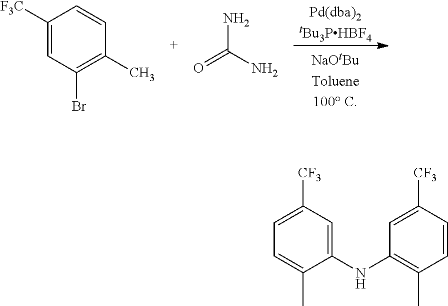

For molecule 4, first in an oven-dried 250 mL Schlenk flask containing 150 mL anhydrous toluene and fitted with a magnetic stirrer bar was added 2-Bromo-1-methyl-4-(trifluoromethyl)benzene (10 g, 42 mmol, 2 eq.), urea (1.3 g, 21 mmol, 1 eq.) and NaO^{t}Bu (12.2 g, 126.95 mmol, 6 eq.) under nitrogen atmosphere and the reaction mixture was sparged with nitrogen for 15 min. After that, Pd(dba)₂ (245 mg, 0.4 mmol, 0.02 eq.) and ^{t}Bu₃·HBF₄ (364 mg, 1.3 mmol, 0.06 eq.) were added to the reaction mixture and the nitrogen sparging continued for 15 min. The reaction mixture was then heated to reflux for 20 hours under nitrogen atmosphere, then allowed to cool to RT. The mixture was diluted with EtOAc and extracted with brine. The organic layer was dried on Na₂SO₄ and concentrated under reduced pressure. The crude product was purified on silica gel column chromatography using petroleum ether as eluent. The fractions containing the pure product were then combined and the solvent was removed via reduced pressure to afford the product as white solid with a yield of 80% (Rr-0.478 in 90:10 petroleum ether/Et₂O).

¹H NMR (400 MHz, CDCl₃): δ=7.33 (d, 2H), 7.21 (d, 2H), 7.15 (s, 2H), 5.33 (s, broad, 1H), 2.3 (s, 6H). ¹³C NMR (100 MHz, CDCl₃): δ=141.62, 131.92, 131.57, 129.52 (q, J=32.4 Hz), 125.62, 122.92, 120.19, 118.78 (q, J=3.9 Hz), 115.04 (q, J=3.9 Hz), 17.95. ¹⁹F-NMR (376.5 MHz, CD₃CN): δ=−62.58 (s).

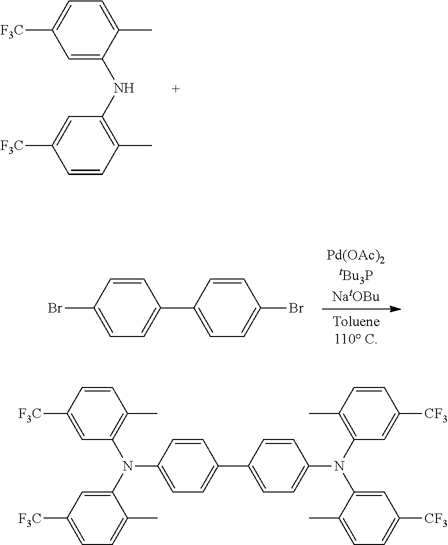 [SMILES: Cc1ccc(C(F)(F)F)cc1Nc1cc(C(F)(F)F)ccc1C ; Brc1ccc(-c2ccc(Br)cc2)cc1 ; Cc1ccc(C(F)(F)F)cc1N(c1ccc(-c2ccc(N(c3cc(C(F)(F)F)ccc3C)c3cc(C(F)(F)F)ccc3C)cc2)cc1)c1cc(C(F)(F)F)ccc1C]

The secondary amine derivative (700 mg, 2 mmol, 2.1 eq.), 4,4′-dibromobiphenyl (312 mg, 1 mmol, 1 eq.) and NaO^{t}Bu (240 mg, 2.5 mmol, 2.5 eq.) were added to a dry round bottom flask containing 5 mL anhydrous toluene under nitrogen atmosphere. The mixture was degassed with a nitrogen stream for 15 min after which Pd(OAc)₂ (9 mg, 0.04 mmol, 0.04 eq.) and ^{I}Bu₃P (6.5 mg, 0.032 mmol, 0.032 eq.) dissolved in 5 mL anhydrous toluene were added and nitrogen purging continued for 10 min. Then, the reaction mixture was heated to reflux and kept stirring overnight under a nitrogen atmosphere. After that, the reaction was allowed to cool down to RT, diluted with EtOAc and extracted three times from water by EtOAc (3×20 mL). The combined organic layers were dried over MgSO₄ and the solvent was removed under reduced pressure. The crude product was purified by silica gel column chromatography using hexanes as eluent. The product was then collected and concentrated via reduced pressure with a yield 90% of white solid flakes.

¹H NMR (400 MHz, CDCl₃): δ (d, J=8.6 Hz, 4H), 7.34 (m, 8H), 7.19 (s, 4H), 6.76 (d, J 8.6 Hz, 4H), 2.05 (s, 12H). ¹³C NMR (100 MHz, CDCl₃): δ=146.62, 146.09, 138.54, 134.11, 132.69, 129.89 (q, J=32.6 Hz), 127.51, 123.65 (q, J=3.8 Hz), 121.82 (q, J=3.8 Hz), 121.55, 19.28. ¹⁹F-NMR (376 MHz, CDCl₃): δ=−62.3

Radical Cations Isolation as PF₆⁻ Salts

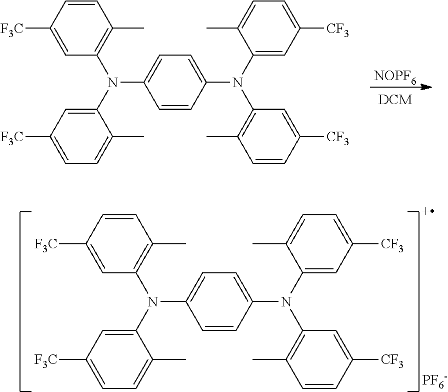

Aryl amine 3 (0.5 g, 0.675 mmol) was dissolved in anhydrous dichloromethane (10 mL) in an oven-dried 25 mL round-bottomed flask fitted with a rubber septum under nitrogen atmosphere. The round-bottomed flask was placed in an ice water bath for 10 min after which nitrosonium hexafluorophosphate (0.130 g, 0.7 mmol) was added into the resultant reaction mixture and stirred 1 h. Upon completion of the reaction, anhydrous diethyl ether (15 mL) was added gradually with continued stirring, resulting in a dark green precipitate. The precipitate was filtered then redissolved in anhydrous dichloromethane (5 mL) and precipitated with anhydrous diethyl ether (10 mL). This process was repeated once more to remove unreacted starting material. The product (0.48 g, 80% yield) was dried under nitrogen and stored in a glove box.

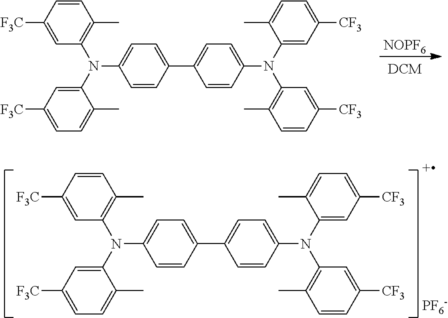

Aryl amine 4 (0.30 g, 0.4 mmol) was dissolved in anhydrous dichloromethane (3 mL) in an oven-dried 5 mL round-bottomed flask fitted with a rubber septum under nitrogen atmosphere. The round-bottomed flask was placed in an ice water bath for 10 min after which nitrosonium hexafluorophosphate (0.071 g, 0.4 mmol) was added into the resultant reaction mixture and stirred 1 h. Upon completion of the reaction, anhydrous diethyl ether (15 mL) was added gradually with continued stirring, resulting in a black precipitate. The precipitate was filtered then redissolved in anhydrous dichloromethane (5 mL) and precipitated with anhydrous diethyl ether (10 mL). This process was repeated once more to remove unreacted starting material. The product (0.114 g, 33% yield) was dried under nitrogen and stored in a glove box.

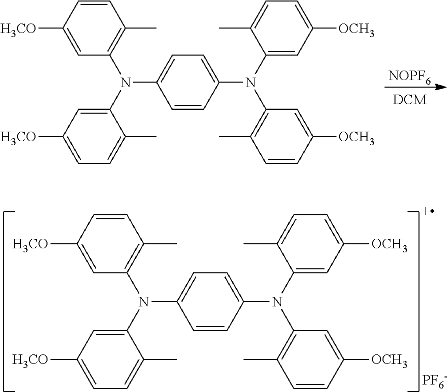

Aryl amine 1 (0.150 g, 0.3 mmol) was dissolved in anhydrous dichloromethane (3 mL) in an oven-dried 5 mL round-bottomed flask fitted with a rubber septum under nitrogen atmosphere. The round-bottomed flask was placed in an ice water bath for 10 min after which nitrosonium hexafluorophosphate (0.54 g, 0.3 mmol) was added into the resultant reaction mixture and stirred 1 h. Upon completion of the reaction, anhydrous diethyl ether (15 mL) was added gradually with continued stirring, resulting in a black precipitate. The precipitate was filtered then redissolved in anhydrous dichloromethane (5 mL) and precipitated with anhydrous diethyl ether (10 mL). This process was repeated once more to remove unreacted starting material. The product (0.136 g, 71% yield) was dried under nitrogen and stored in a glove box.

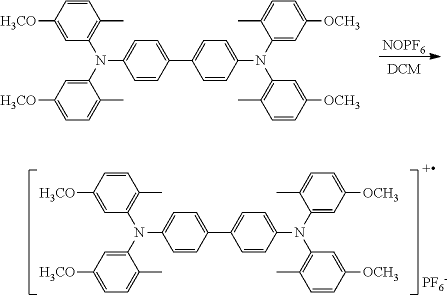

Aryl amine 2 (0.5 g, 0.8 mmol) was dissolved in anhydrous dichloromethane (3 mL) in an oven-dried 5 mL round-bottomed flask fitted with a rubber septum under nitrogen atmosphere. The round-bottomed flask was placed in an ice water bath for 10 min after which nitrosonium hexafluorophosphate (0.158 g, 0.90 mmol) was added into the resultant reaction mixture and stirred 1 h. Upon completion of the reaction, anhydrous diethyl ether (15 mL) was added gradually with continued stirring, resulting in a black precipitate. The precipitate was filtered then redissolved in anhydrous dichloromethane (5 mL) and precipitated with anhydrous diethyl ether (10 mL). This process was repeated once more to remove unreacted starting material. The product (0.360 g, 71% yield) was dried under nitrogen and stored in a glove box.

### Cyclic Voltammetry

CV measurements were carried out in a nitrogen-filled glovebox at ambient glovebox temperature using a VSP-300 potentiostat (BioLogic). Unless otherwise indicated, the CVs were recorded in 0.2 M TBAPF₆/CH₃CN as electrolyte with 3 mm diameter glassy carbon (CH Instruments, Inc.) as the working electrode. A non-aqueous Ag/Ag⁺ (silver wire immersed in CH₃CN containing 0.1 M TBABF₄ and 0.01 M AgBF₄) electrode was used as the reference electrode, and a Pt wire (CH Instruments, Inc.) as the counter electrode. Before each measurement, glassy carbon electrodes were mechanically polished with an aqueous slurry of graded alumina (0.05 μm), then ultra-sonicated in water then acetone for 1 min each. Voltammograms were recorded at a scan rate of 100 mV·s⁻¹. Ferrocene was used as an internal reference and the oxidation potentials were calibrated with respect to ferrocene/ferrocenium (Cp₂Fe^{0/+}) redox couple. The diffusion coefficients were determined using Randles-Sevick equation:

$${i}_{p} = 0.4463 {nFAc ( \frac{nFvD}{RT} )}^{\frac{1}{2}}$$

with R being the perfect gas constant (8.314 J·mol⁻¹·K⁻¹), F the Faraday's constant (96485 C·mol⁻¹), T the absolute temperature (˜298 K), iₚ is the peak current intensity (in A), n the number of transferred electrons, A the area of the electrode (cm²), D the diffusion coefficient (cm²·s⁻¹), v the scan rate (V·s⁻¹) and c the concentration (mol·cm⁻³). Data obtained from voltammograms at scan rates of 25, 50, 75, 100, 200, 300 and 400 mV·s⁻¹ were used for the calculation of diffusion coefficients.

### Solubility Determination

The solubility of the compounds in pure acetonitrile was determined by the mass measurement method described previously. Saturated solutions of each compound were prepared and filtered to remove any undissolved material. Afterwards, 3 mL of each solution were taken and added to previously weighed vials (the measurement was repeated three times for each solution, i.e. 3 vials for each solution). The vials were then stored open in a nitrogen-filled glove box to allow the slow evaporation of the solvent. After 10 days, acetonitrile was evaporated from all vials, leaving only the previously dissolved solid. The vials were then re-weighed and the mass of the solid was determined by subtracting the mass of the respective empty vial. The concentration of the compound was calculated from the mass of the dissolved solid and the volume of the sample placed into the vial.

### Bulk Electrolysis:

Essentially, if trace impurities existing in the electrolyte trigger self-discharge of the charged species, a relatively large portion of oxidized species will switch to its uncharged form if the concentration of active species is low. However, at elevated concentrations, the relative concentration of impurities with respect to active species will be considerably lower and their effect will be minimized. In other words, higher CEs are expected when the active species concentration is larger. Electrochemically oxidizing the entire solution (charging) and subsequently reducing it (discharging) resulted in varied CEs depending on the identity of the involved catholyte. CE is calculated by dividing the amount of charge passed during discharging (Q_{discharge}) by the amount of charge passed during charging (Q_{charge}). A considerably lower than 99% CE indicates instability and decomposition of active material via parasitic processes. It is known that CE is affected by the C-rate at which the battery is charged/discharged. Higher C-rates usually yield higher CEs since less time is given for unwanted parasitic processes to consume the charged species, unlike in slow C-rates where such processes are allowed to consume charge leading to lower CEs. On the other hand, in the ideal case, if the charged form of the active species is both chemically stable and does not undergo self-discharge, the CE should not change with changing C-rate.

BE experiments were performed in custom-made H-cells. The H-cell has two electrolyte chambers separated by an ultra-fine porous glass frit (P5, Adams and Chittenden) which prevents the mixing of the electrolytes. Two homemade reticulated vitreous carbon (45 PPI, Duocel®) electrodes, placed in both chambers separately served as the working and counter electrodes. A non-aqueous Ag/Ag⁺ (silver wire immersed in CH₃CN containing 0.1 M TBABF₄ and 0.01 M AgBF₄) electrodes was used as a reference electrode and was placed next to the working electrode in the same chamber. In addition, a 3 mm-glassy carbon electrode (CH Instruments, Inc.) was attached to the working electrode chamber to record CVs before and after bulk electrolysis experiments. In each of the working and the counter electrode chambers, 3.5 mL of electrolyte containing a given concentration of the neutral active species and 0.1 M TBAPF₆/CH₃CN were added to both the working and the counter electrode chambers for charge-discharge cycling. The voltage cut-offs were determined by recording a CV in the working electrode side prior to the start of the bulk electrolysis experiment. The values of the cut-off voltage were set by adding 0.3 V to the calculated half-way oxidation potential determined by CV. Both the working and the counter electrode chambers were stirred throughout the bulk electrolysis experiments at a stirring speed of 900 rpm to maintain mass transfer during charging and discharging. The experiment was conducted at variable C-rates, starting with 3 C then 2 C and finally 1 C with 8 cycles run at each C-rate. The galvanostatic control and data acquisition were achieved by incorporating a BioLogic VSP potentiostat.

### UV-vis and UV-NIR Spectroscopy

In an argon-filled glove box, 0.5 mM solutions of neutral and radical cation were prepared in acetonitrile and pipetted into 1 mm path length optical glass cuvettes. The choice of this path length of cuvette is dictated by the aim to prevent the UV-vis detector saturation of the intensely colored cationic species. All samples destined to UV-vis stability monitoring contained 0.1 M TBAPF₆ whereas UV-NIR samples were in pure CH₃CN. Solutions were added to the cuvettes inside an argon filled glovebox, sealed with a Teflon screw cap and removed from the glovebox for spectral analysis. UV-vis spectra were collected in an Agilent 8453 diode-array spectrometer. Spectra were collected at 0, 1, 3, 4, 5, 6 and 24 h after dissolution.

### UV-Vis Spectro-Electrochemistry

UV-vis spectro-electrochemical data was obtained by running a 30-minute constant potential bulk electrolysis at the oxidation potentials determined from cyclic voltammetry to generate respective oxidation states for each molecule. A CHI 1100B potentiostat with graphite rod working and counter electrodes and a non-aqueous Ag/Ag⁺ reference electrode were used to perform the electrolysis on solutions of 1 mM of each molecule contained in acetonitrile and 0.1M TBAPF₆ electrolyte. These solutions were diluted to 1×10⁻⁵ M for spectral analysis with an Ocean Optics UV-vis spectrometer in a 1 cm quartz cuvette.

### Symmetric Flow Cell Cycling

A custom made, small volume custom flow cell with interdigitated flow fields was used in this work as used previously in refs. The 2.55 cm² flow cell consisted of two compartments separated by a 175 m thick Daramic membrane. 10 mL of equimolar solution (total concentration of 10 mM) of compound 3 and its PF₆ salt was used in each reservoir (Savillex) initially. The electrolyte was circulated at 10 mL min⁻¹ using peristaltic pump (Masterflex L/S Series) equipped with norprene tubing (Masterflex). Capacity retention study was performed and data was collected using a VSP potentiostat (BioLogic). The flow cell was then cycled for 200 cycles at a current density of 5 mA cm⁻² with potential cut-offs of ±0.45 V. Electrochemical impedance spectroscopy (EIS) was collected using the same instrument before and after cycling at the open-circuit potential with a sinusoidal amplitude of 10 mV between 200 kHz and 10 mHz (5 points per decade).

### Quantum-Chemical Calculations

All quantum-chemical calculations were carried out with density functional theory (DFT) at the (IP-tuned) LC-ωHPBE/Def2SVP level of theory via the Gaussian16 (rev A.03) software suite. For each molecule investigated, the long-range correction parameter o is tuned via an iterative ionization potential (IP) tuning procedure described by Baer and Kronik; the tuned functional was then used for all subsequent calculations. Calculations that account for the impact of the solvent dielectric were performed with the implicit polarized continuum model (PCM), using acetonitrile with a dielectric constant F of 35.688. All measurements of geometry and FMOs were made with structures optimized with implicit solvation. Additionally, all spectroscopy data were generated with time-dependent DFT (TD-DFT) with implicit solvation. Oxidation potentials estimations used the Born-Haber cycle method. Reorganization energies (λ) are calculated with the four-point method.

Density functional theory (DFT) calculations at the (IP-tuned) LC-ωHPBE/Def2SVP level of theory in implicit solvent were carried out to determine the natures of the neutral, radical-cation, and dication states of 1-4. In this discussion, we focus on the neutral and radical-cation states because shortcomings in solvent considerations make the dication calculations less reliable. The neutral states, as expected, have both geometric and electronic inverse symmetry for all molecules. The symmetry centers around the phenyl (1 and 3) or biphenyl (2 and 4) bridge for each molecule. The biphenyl-bridged systems have a dihedral twist of 330 in 2 and 340 in 4. Also, for every neutral molecule, the three nitrogen bonds on each redox center are mostly trigonal planar, though some are slightly pyramidal. The sums of the angles around each nitrogen are 358.4°±1.8°. While for each molecule the redox centers on each side are symmetric with relation to the bridge, there is some difference for molecules 1 and 2 versus molecules 3 and 4. The average dihedral angle between the biphenyl or phenyl bridge and the redox-center nitrogen (on both sides) is 34°±3° for the methoxy-substituted redox centers of 1 and 2 and 27°±1° for the CF₃- and methyl-substituted redox centers of 3 and 4. The electronic structures for these systems are similarly symmetric. For each molecule, the HOMO-1, HOMO, and LUMO are delocalized throughout the phenyl (1 and 3) or biphenyl (2 and 4) bridge and both diaryl amine redox centers. Notably, the nitrogen p-orbitals are antisymmetric for the HOMO and symmetric for the HOMO-1, representing the two linear combinations as expected.

The solubilities of 1-4 were examined in pure CH₃CN using the mass measurement method. While molecules A and B, the non-substituted analogues of molecules 1 and 2 are virtually insoluble in CH₃CN, the modification on the framework by grafting methoxy groups enhances the solubility to 9 mM and 3.5 mM, respectively (Table 1). Interestingly, the fluorinated molecules show a further solubility enhancement of 13 mM for both 3 and 4.

### Isolation of Charged Forms (Radical Cation) Via Chemical Oxidation

The radical cations 1^{+⋅}, 2^{+⋅}, 3^{+⋅} and 4^{+⋅} of 1, 2, 3 and 4, respectively, were synthesized as PF- salts by chemical oxidation using nitrosonium hexafluorophosphate (NOPF₆). The use of nitrosonium salts is particularly convenient as the reduction product of these reagents is a gas (NO), implying that no particular purification step is needed at the end of the reaction, since no chemical reagents remain after the radical cation forms. The synthetic procedure is described in the Experimental Section and the presence of the various radical cations was confirmed using Electron Paramagnetic Resonance (EPR) spectroscopy. In EPR spectroscopy, only paramagnetic species (such as radicals) generate a signal in response to the applied magnetic field, while diamagnetic species (such as neutral and dicationic forms of bis-TPA investigated here) that don't possess unpaired electrons remain EPR silent.

DFT calculations show neither electronic nor geometric symmetry breaking for the radical-cations. Instead, the radical-cation structure for each system generally flattened when compared to the neutral geometries. All the redox-center nitrogens shift to very trigonal planar geometries, with sums of the angles around each nitrogen shifting to 359.7°±0.5°. Additionally, the dihedral angles between the biphenyl or phenyl bridge and the redox-center nitrogens decreased for almost every redox center, and the dihedral twist for the bridging biphenyls decreased to 25° in molecule 2 and 13° in molecule 4. There is also no charge localization in the DFT-computed radical-cation frontier molecular orbitals (FMOs). The planar geometries and electronic symmetry across all radical-cations (instead of one redox center possessing more charge and/or a distorted geometry) suggests that the oxidation charge is delocalized fully throughout each system. This indicates that these systems are all Robin-Day class III mixed-valence system with high electronic coupling (discussed in more detail later). Nevertheless, the DFT used here likely over-delocalized the electron density especially in mixed valence systems due to the multi-electron self-interaction error, which leaves open the possibility that these are borderline class II/III systems.

The synthesized cations were also characterized using UV-NIR spectroscopy (FIG. 3). From FIG. 3 it can be clearly seen that the spectra of the charged forms change dramatically as compared to the neutral forms. Notably, the solutions of the cations were very intensely colored whereas the neutral forms solutions ranged from colorless to very weak and transparent colors depending on the molecule. All cations exhibit a remarkably strong absorption peak in the NIR region. This observation is expected, as these particular peaks are characteristic of species that possess mixed-valence characteristics. A and B shows the emergence of inter-valence charge transfer (IVCT) bands centered at ˜10000 cm⁻¹ and ˜7500 cm⁻¹ for 1^{⋅}. and 2^{+⋅}., respectively. These values match perfectly to those reported by Lambert and Noll for the same molecules using spectroelectrochemistry in 0.1 M TBAPF₆ in dichloromethane.²¹

Spectra determined through time-dependent DFT (TD-DFT) calculations at the (IP-tuned) LC-ωHPBE/Def2SVP level of theory agree generally well with the experimental UV-Vis data. The dominant transition for each neutral species is the HOMO □ LUMO transition. With the exception of 1, this dominant HOMO □ LUMO transition is the lowest energy transition. For 1, the dominant transition is the second lowest energy transition, a HOMO □□LUMO excitation, similar to all the other dominant neutral transitions. All of these transitions consist of about 55-65% HOMO □□LUMO excitation while the remaining contributions are mostly excitations from the HOMO to higher unoccupied orbitals. Each of the radical-cation and dication TD-DFT calculations produces a strong transition in the near-IR range that corresponds to the experimentally observed IVCT band, though the TD-DFT tends to underestimate the transition energy compared to experimental data. In each radical-cation, this lowest energy transition corresponds (with over 90% configuration contribution) to the excitation of an electron to the single occupied molecular orbital (SOMO, the analog to the neutral species HOMO) from the radical cation analog for the HOMO-1. In each dication, this lowest energy transition corresponds to the excitation of an electron from the HOMO to the LUMO.

### Cyclic Voltammetry

Electrochemical characterization of 1-4 was achieved by cyclic voltammetry (CV) in 0.1 M TBAPF₆ in CH₃CN. Glassy carbon was used as the working electrode and 1 mM of active materials were used. The resulting voltammograms are shown in FIG. 4. All derivatives featured two distinct one-electron oxidation waves upon voltage sweep. The first and the second waves correspond to the sequential neutral to radical-cation and radical-cation to dication transformations, respectively. These features are in line with the presence of two redox centers in these molecules. The redox peaks associated with the radical cation formation for molecules 1 and 2 are centered at 0 and 0.17 V vs Fc⁺/Fc, respectively (FIG. 4). These values are in line with the data reported in literature for the same molecules.⁸ Note that in previous literature the electrochemistry is reported in 0.1 M TBAClO₄ in dichloromethane and reported versus a Ag/AgCl reference electrode. The Fc⁺/Fc couple has a redox potential at 0.403 V vs SCE and 0.44 V vs Ag/AgCl reference electrode. On the other hand, molecules 3 and 4 feature the radical cation transformation redox peaks at 0.41 and 0.53 V vs Fc⁺/Fc, respectively (FIG. 4).

The presence of highly electronegative —CF₃ groups provokes a considerable increase in the oxidation potentials in 3 and 4 compared to 1 and 2. By the same principle, the presence of electron donating groups such as the methoxy group contributes to lowering the oxidation potential compared to the parent unsubstituted molecules. DFT-calculated oxidation potentials reflect the experimental trends well. Additionally, DFT-derived adiabatic ionization potentials (AIP) estimate the energy required to extract an electron without solvation effects. Here, systems with CF₃ and methyl groups (5.84 eV for 3 and 8.01 eV for 4) are significantly higher than the AIPs for the molecules with methoxy groups (4.69 eV for 1 and 5.24 eV for 2). This supports the hypothesis that the electronegative —CF₃ groups increase the energy required to extract an electron, resulting in higher oxidation potentials. When the current peaks were traced against the square root of applied scan rates, a linear relationship is established for each compound, confirming a diffusion-controlled mass transport as inferred from the Randles-Sevcik equation. The diffusion coefficients of each compound were calculated at 1 mM concentration using the Randles-Sevcik equation. The diffusion coefficients of 1 and 3 are larger than those of 2 and 4, which is expected since 1 and 3 are smaller in size.

TABLE 1
Measured redox potentials, diffusion coefficients
of 1 mM of compounds 1, 2, 3 and 4 in 0.2M TBAPF₆
/CH₃CN and their solubilities in pure acetonitrile.

| | | | ΔE (E^{o′2} − | | | |
|---|---|---|---|---|---|---|
| Mol. | E^{o′1 [a]} | E^{o′2 [a]} | E^{o’1}) | K_{c} | D ^{[b]} | Solubility^{[c]} |
| 1 | 0.00 | 0.38 | 0.38 | 3 × 10⁶ | 4.49 | 8.76 |
| 2 | 0.17 | 0.33 | 0.16 | 5 × 10² | 1.81 | 3.50 |
| 3 | 0.41 | 0.91 | 0.50 | 3 × 10⁸ | 7.67 | 13.34 |
| 4 | 0.53 | 0.69 | 0.16 | 5 × 10² | 1.54 | 13.00 |

^{[a]} in V vs Cp₂Fe^{0/+}.
^{[b]} Diffusion coefficient in 10⁻⁶ cm² · s⁻¹.
^{[c]}in acetonitrile given in mM.

The bridging moiety between the two nitrogen centers has a considerable impact on the electrochemical behavior of the molecule. More precisely, the CV of the molecules constituting of one phenyl ring as a bridge between the two redox centers, i.e. derivatives 2 and 4, exhibit two well-separated oxidation waves. On the other hand, the wave-to-wave separations in CV of the molecules with two phenyl rings in their linker are remarkably smaller. The reason for such a discrepancy can be attributed to the difference in the degree of electronic communication between the two arylamine moieties being weaker in the molecules having biphenyl as a bridge.

In fact, these molecules belong to a class of compounds known as mixed-valence (MV) compounds. Mixed valency is a term that refers to a chemical species, whether organic or inorganic, with two or more redox centers that have different oxidation stated. This concept was first introduced in 1915 by Hofmann and Hoeschele. MV species are usually intensely colored due to the unique electronic transitions associated with intervalence electron transfer. CV is a useful technique in studying MV species since it provides insights on how stable one ionic species will be with respect to disproportionation, using the comproportionation constant (K_{c}). K_{c} is used to measure the ease of isolating the radical-cation rather than obtaining a mixture of neutral, radical-cationic, and dicationic species. K_{c} can be calculated from the ΔE which is the value of the redox potential difference between the first and the second oxidation events. K_{c} is a quantity used to evaluate the electronic communication between two redox centers in mixed-valence species. Large comproportionation constant, i.e. a substantial value of ΔE, is a crucial requirement for the isolation of mixed-valence species separately of other oxidation states of the species. K_{c} is defined as:

$${K}_{C} = {e}^{\frac{Δ {En}_{1} {n}_{2} F}{RT}}$$

where F is the Faraday constant, R is the gas constant, n₁ and n₂ are the numbers of electrons transferred in each redox process and T is temperature in Kelvin. In the typical case where n₁=n₂=1, the equation simplifies to:

$${K}_{C} = {e}^{\frac{Δ E}{25.69}}$$

at 298 K with ΔE given in mV.

In this work, molecules 1 and 3 have considerably larger Kc values than 2 and 4 (Table 1) due to the ΔE originating from the large separation of the two oxidation waves observed in CV (FIG. 4). This highlights the significance of the spacer moiety in controlling the electron transfer properties between redox centers within MV species. Moreover, this means that isolating the monocationic species should be easier in 1 and 3 than in 2 and 4. This will have an impact on the electrochemical performance of any RFB involving MV compounds as active species such as the number of transferred electrons at a defined voltage and/or the stability of the charged state of the active species (monocationic vs dicationic).

According to Robin and Day, the MV species are classified based on the electronic coupling between the redox centers into three classes, I, II and III. In Class I species, no electronic coupling exists between the redox centers. In Class II, localized valences with measurable electronic coupling exist and gives rise to an intervalence charge transfer (IVCT) band and a thermal barrier to electron exchange. In Class III, the redox centers have a non-integral valence and are indistinguishable, the lone electron/hole is delocalized equally over the redox sites.

Class II or III assignment is based on electronic spectroscopy; the broadness and line shape of the IVCT transition is analyzed in this case. Analysis of the line broadness and line shape of the IVCT band is also used to determine whether a mixed-valence species belongs to Class II or III. Hush theory provides a relationship that simplifies at 298 K to:

$${v}_{\frac{1}{2}} = \sqrt{2310 {v}_{max}}$$

where vₘₐₓ is the maximum absorption of the IVCT band given in cm⁻². If a Gaussian-shaped band has a v_{1/2} larger than that predicted by this relationship, this result is suggestive that the IVCT band belongs to a Class II species. If the band is narrower than that predicted, this result is suggestive of a Class III species. For all molecules studied in this work, the measured v_{1/2} is smaller than the Hush-predicted v_{1/2} (Table 2), indicating that all are Class III systems.

TABLE 2
Measured spectroscopy data (νₘₐₓ and ν_{1/2}) along with
Hush-theory ν_{1/2}. All values given in cm⁻¹.

| Mol. | νₘₐₓ | ν_{1/2} | ν_{1/2, Hush} |
|---|---|---|---|
| 1 | 9790 | 3846 | 4756 |
| 2 | 9728 | 3524 | 4740 |
| 3 | 12849 | 4018 | 5448 |
| 4 | 8238 | 2519 | 4362 |

Trends in electronic coupling can also reflect where localization is occurring in experiments. Lower electronic coupling characterizes class II/III molecules. Several methods of calculating electronic coupling using both experimental and computational descriptors show that biphenyl-bridged molecules (2 and 4) exhibit smaller electronic coupling than the phenyl-bridged molecules (1 and 3). While the biphenyl HOMO is higher than that of the phenyl bridge which might allow for greater coupling, the 9.28 Å biphenyl bridge (relative to the 4.97 Å phenyl bridge) increases the spatial distance between redox centers, leading to the smaller coupling (FIG. 5). Analytically, class II and III molecules are delineated by comparing the electronic coupling (H_{ab}) and Marcus reorganization energy (λ): ²H_{ab}<λ for class II, and 2H_{ab}>λ for class III. Here, electronic couplings are estimated as half the gap between the HOMO and HOMO-1, and reorganization energies are calculated with the four-point method. While 2H_{ab} is greater than λ for all systems, the margin is much smaller for the biphenyl-bridged systems (FIG. 5). This indicates that the biphenyl-bridged 2 and 4 may be class III systems, but they are closer to the II/III borderline than the phenyl-bridged 1 and 3. This is reflected in the two redox events at similar potentials present in the cyclic voltammograms for molecules with the biphenyl bridge.

### Spectro-Electrochemistry

The UV-NIR spectra of the chemically synthesized cations were corroborated by UV-vis spectro-electrochemistry to study the electronic properties of the neutral and charged species for each molecule (FIG. 6).

The respective radical-cation and dication species were generated by performing bulk electrolysis at the oxidation potentials determined from cyclic voltammetry for each molecule. Each charged species was analyzed spectroscopically, which provided comparison to UV-vis spectra obtained from chemical oxidation studies (FIG. 4). Spectra for all neutral molecules match the neutral spectra acquired by dissolving these at 0.5 mM in 0.1 M TBAPF₆/CH₃CN. The spectra of 1, 2, 1^{+⋅}, 2^{+⋅}, 1²⁺ and 2²⁺ obtained in this study agree with reported data in literature. For molecules 1, 2, and 3, the spectra for the electrochemically generated radical-cation species is similar to the radical-cation generated through chemical oxidation. The only discrepancy for 4 is the absence of the peak around 600 nm that is present in the chemical oxidation spectrum and the remainder of a portion of the peak around 350 nm. There is likely some equilibrium occurring between charged species for 4 stemming from its high oxidation potentials and the proximity of redox features to one another that impacts its UV-vis spectrum. This is also observed in later UV-vis stability studies.

Comparing the radical-cation and dication spectra for each species, there is a distinct change in the spectra for molecules 1 and 2 that is not observed for molecules 3 and 4. The main difference between molecule types stems from the substituents present on the aryl groups. It appears the methoxy groups in 1 and 2 lead to unique electronic transitions for each oxidation state, whereas the CF₃ and methyl groups on 3 and 4 only lead to unique electronic transitions between charged and neutral states. It is possible that the strong electron donating effect from the aryl methoxy groups on 1 and 2 introduces stabilizing electronic effects that provide distinct UV-vis spectra for each charge state. The strongly electron withdrawing CF₃ and weakly donating methyl groups on 3 and 4 do not have this same donating capability, which only leads to change in UV-vis spectra between neutral and any charged state.

### Stability Via UV-Vis Spectroscopy and Bulk Electrolysis

UV-vis spectroscopy provides a means to monitor the stabilities of the radical cations over prolonged time scales that extend beyond that offered by CV. This is possible since all the charged forms show absorption features in the UV and visible regions, which enables monitoring of the change of concentrations over the course of time, based on changes in peak intensities.

The radical cation samples were prepared in 0.1 M TBAPF₆/CH₃CN inside an argon-filled glovebox and transferred into sealed cuvettes to prevent contact with atmospheric moisture/air when taken outside the glovebox. It is worthwhile to mention that due to the high extinction coefficients of the radical cations particularly at longer wavelengths, a quartz cuvette of 1 mm pathlength was used in order to prevent detector saturation with 0.5 mM sample concentration. It is also worth mentioning that we identified 0.5 mM as an optimal concentration for UV-vis spectroscopy studies, because higher concentrations led to detector saturation and inaccuracy of the measurements. Higher concentrations in such studies would be advantageous to minimize the impact of trace impurities present in the supporting electrolyte or the solvent on the overall stability of the charged species. This also better mimics the experimental conditions in a RFB where higher concentrations are used.

FIG. 7 shows the absorption spectra for 0.5 mM of the various radical charged forms of compounds 1, 2, 3 and 4 in 0.1 M TBAPF₆/CH₃CN over a period of 24 h. The neutral samples of each molecule were also monitored for the sake of comparison. Per a previous study, the emergence of new absorption peaks in the UV-vis spectrum would likely be due to the formation or cleavage of covalent bonds, producing new chemical species that have different electronic absorptions. Changes in the intensity of the peaks could arise either from reduction of charged species to their neutral forms through an electron transfer (self-discharge) mechanism or from active species decomposition.

The UV-vis spectra of all the neutral forms remained virtually identical over the course of 24 hours, highlighting the solution stability of these materials under such conditions. Apart from 4+., the spectra of the cations show highly stable absorption spectra with very subtle changes observed at shorter wavelengths in the UV-vis spectrum of 3+. The rapid decay observed in the UV-vis spectrum of 4+. points to the chemical instability of this molecule in its charged form. This instability is also evident during the electrochemical galvanostatic cycling as will be shown later below. A reason for such instability could be due to the predominant presence of the dicationic (+2) form in the sample compared to the radical cation (+1) form, which is expected to exhibit higher instability. In other words, the UV-vis spectrum in D is most likely showing the absorption bands of a mixture of the radical-cationic (absorption at ˜450 nm) and the dicationic species (absorption at ˜650 nm). The band of the dicationic species at ˜650 nm decays rapidly at the expense of the growth of the band of the presumably radical-cationic species at ˜450 nm. This is supported by the UV-vis spectrum obtained by spectro-electrochemistry where the absorptions of the +1 and +2 forms are well-resolved. Attempts to isolate the radical-cationic species by chemical oxidation were not successful and a mixture of the two charged forms was obtained at each time. This could be rationalized if we consider the proximity of the two oxidation events observed by CV and the resulting small comproportionation constant

The ordering of stabilities from highest to lowest observed here matches the ordering of the oxidation potentials of TPA derivatives from least to greatest. In other words, the radical cations derived from catholytes with higher oxidation potentials exhibit faster decay in absorption intensity. These results show that these cations are stable in solution, and that minimal self-discharge or decomposition is taking place during storage in the charged and neutral states. It is worth mentioning that at higher concentrations like those used in RFBs, the already high stability will be reinforced as illustrated previously.

Bulk electrolysis (BE) can reveal the presence of side reactions during extended time of charging/discharging. These are difficult to detect by CV due to the relatively short time scales of such measurements. Moreover, the fact that bulk electrolysis involves electrolyzing large volumes of solutions, gives it more significance as it provides a better approximation of the working conditions of RFBs. On the contrary, in CV measurements, only a negligible volume of solution-confined to the thin diffusion layer nearby the electrode surface—is involved in the electrochemical reaction.

Galvanostatic bulk electrolysis (BE) cycling was performed to monitor the charged species self-discharge by following the coulombic efficiency (CE). BE is particularly interesting because it provides deeper insights on the electrochemical stability of the electroactive compounds upon continuous galvanostatic cycling. More information about this technique is provided in the supplementary information. Essentially, if trace impurities existing in the electrolyte trigger self-discharge of the charged species, a relatively large portion of oxidized species will switch to its uncharged form if the concentration of active species is low. However, at elevated concentrations, the relative concentration of impurities with respect to active species will be considerably lower and their effect will be minimized. In other words, higher CEs are expected when the active species concentration is larger.

Electrochemically oxidizing the entire solution (charging) and subsequently reducing it (discharging) resulted in varied CEs depending on the identity of the involved catholyte. CE is calculated by dividing the amount of charge passed during discharging (Q_{discharge}) by the amount of charge passed during charging (Q_{charge}). A considerably lower than 99% CE indicates instability and decomposition of active material via parasitic processes. It is known that CE is affected by the C-rate at which the battery is charged/discharged. Higher C-rates usually yield higher CEs due to the fact that less time is given for unwanted parasitic processes to consume the charged species, unlike in slow C-rates where such processes are allowed to consume charge leading to lower CEs. On the other hand, in the ideal case, if the charged form of the active species is both chemically stable and does not undergo self-discharge, the CE should not change with changing C-rate.

FIG. 8 shows the BE data of 1, 2, 3 and 4 carried out in a nitrogen filled glovebox at room temperature. BE cycling was performed at three different C-rates 3 C, 2 C, and 1 C with eight charge/discharge cycles at each C-rate. At slower charge-discharge rates, the accessed capacities were higher. This is expected due to the increase in the cell polarization at faster cycling rates.

The coulombic efficiency during BE of 3 (FIG. 8C) ranged from −97 to 100% per cycle, with a capacity retention of 99%, 97% and 94% after each 8 cycles at 3 C, 2 C and 1 C, respectively. The slight capacity fade observed may be attributed in part to species crossover across the porous frit that separates the counter-electrode chamber from the working-electrode chamber of the bulk electrolysis cell. FIG. 9 shows the cyclic voltammograms of the various molecules performed before and after the BE experiment to diagnose their electrochemical stability. CVs of the solution of 3 before and after BE show nearly identical features, indicating the absence of material decomposition and confirming that 3 is stable upon cycling back and forth between its neutral and radical cation forms. Only a slight decrease in peaks intensity was observed, consistent with the capacity fade that occurs during cycling (FIG. 9C). These results demonstrate the stability of 3 in its neutral and radical-cation forms under this electrochemical environment.

The analogous molecule 1 (FIG. 8A) exhibited excellent BE behavior with high capacity retention (˜99% at all C-rates) and high CE that fluctuated between 99 and 99.9% throughout the cycling. The nearly identical cyclic voltammograms before and after the BE experiment indicates the absence of any decomposition or cross-over during cycling, highlighting the high stability of this molecule (FIG. 9A).

During the BE of 4 (FIG. 8D) a remarkable capacity decay was observed at all C-rates at which the cycling was performed. At faster C-rates the CE was higher, which implies that faster charging/discharging allows less time for self-discharge to take place. The accessed capacity is slightly higher than the theoretical capacity set for one electron transfer process. This means that during the BE cycling, a fraction of the material is oxidized to the dicationic form and adds more capacity on top of the targeted capacity. In fact, this access to the second oxidation process couldn't be avoided in our experimental conditions (galvanostatic BE) although the cut-off voltage was set for this purpose. If one looks back to the comproportionation constant and the small wave-to-wave separation for 4, this result is supported. This same phenomenon is also encountered with molecule 2, which has the same small wave-to-wave separation and two aryl rings in its bridge. However, molecule 4 suffers from a considerable capacity decay that is likely in addition to the cross-over phenomenon, which is attributed to chemical decomposition supported through the substantial decrease in the peak heights of the CV recorded following BE and the emergence of new features between 1 and 1.4 V (FIG. 9D). Molecule 2 does not show any evidence of decomposition with identical CVs recorded before and after BE (FIG. 9B). In addition, 2 maintained good capacity retention and CE throughout the BE cycling (FIG. 8B). The reason for such a difference between the two molecules may be attributed to the difference in their oxidation potentials, with 4 having a considerably higher oxidation potential (0.53 V vs Fc/Fc), compared to 2 (0.17 V vs Fc/Fc). At higher oxidation potentials, more parasitic reactions and chemical decomposition pathways may be promoted leading to the loss of charged material. It was previously observed that the charged species derived from the low oxidation potential catholytes are persistent over time compared to charged species derived from higher oxidation potential catholytes.

### Redox Flow Cell Cycling

Based on the chemical and electrochemical stability results shown above and the oxidation potentials of all molecules, compound 3 is a good initial candidate for implementation in a RFB. For the proof of concept, a symmetric flow cell was used to study the cycling stability of our newly developed catholyte molecule in its neutral and charged form. An equimolar mixture of compound 3 and its corresponding hexafluorophosphate salt (FIG. 10A) was initially used in both the compartments of the flow cell. Hence, the cell was assembled at 50% state-of-charge (SOC) and impedance spectrum was recorded prior to cycling using electrochemical impedance spectroscopy (EIS). The ohmic contribution (5.5 Ωcm²) estimated from the high frequency intercept of the Nyquist plot is consistent with other non-aqueous studies using Daramic separator.

Since compound 3 is soluble to −13 mM, we conducted cycling studies at 10 mM (in 0.1 M TBAPF₆/CH₃CN) and used a current density of 5 mA cm⁻². The cell accessed 60% of the theoretical capacity (2.68 mAh) in the first cycle. The lower accessed capacity may be due to mass transport limitations and can be optimized using lower current density. Though the accessed capacity was lower, the capacity remained stable for 200 cycles (FIG. 10B). The cell retained 97% and 95% of first cycle capacity after 100 and 200 cycles respectively. Slow capacity fade may be because of presence of trace impurities in electrolyte. Overall, flow cell cycling of compound 3 proceeds with average 97% coulombic efficiency over 200 cycles. These preliminary results indicate good cycling stability of these compounds. Future efforts are focused on achieving higher molar concentrations of these compounds in non-aqueous electrolytes.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. A arylamine compound comprising a structure as set forth in Formula I and/or II: 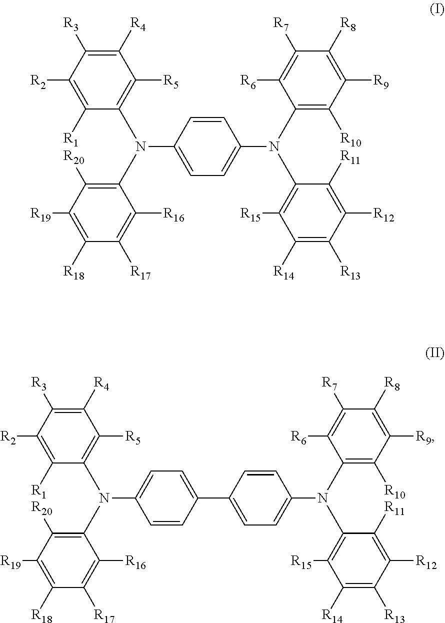
wherein: any of R₁ to R₂₀ are independently hydrogen, methoxy, trifluoromethyl, methyl, or diethylene glycol monomethyl ether. In some aspects, the central phenyl(s) may be appended with a further functional group, such as a methoxy group. In some aspects, two central phenyl rings of Formula II are separated by an intermediary alkyl chain, such as a methyl group.

2. The compound of claim 1, wherein all of R₁ top R₂₀ are hydrogen.

3. The compound of claim 1, wherein R₃, R₈, R₁₃, and R₁₈ are methoxy.

4. The compound of claim 3, wherein all further R groups are hydrogen.

5. The compound of claim 1, wherein R₂, R₉, R₁₂, and R₁₉ are trifluoromethyl.

6. The compound of claim 5, further wherein and R₃, R₈, R₁₃, and R₁₈ are methyl.

7. The compound of claim 1, wherein R₂, R₉, R₁₂, and R₁₉ are trifluoromethyl.

8. The compound of claim 7, further wherein R₅, R₆, R₁₅, and R₁₆ are methyl.

9. The compound of claim 1, wherein R₃, R₈, R₁₃, and R₁₈ are trifluoromethyl.

10. The compound of claim 9, further wherein R₅, R₆, R₁₅, and R₁₆ are methyl.

11. The compound of claim 1, wherein R₄, R₉, R₁₄, and R₁₉ are trifluoromethyl.

12. The compound of claim 11, further wherein R₃, R₈, R₁₃, and R₁₈ are diethylene glycol monomethyl ether.

13. The compound of claim 1, wherein the compound is selected from the group consisting of:

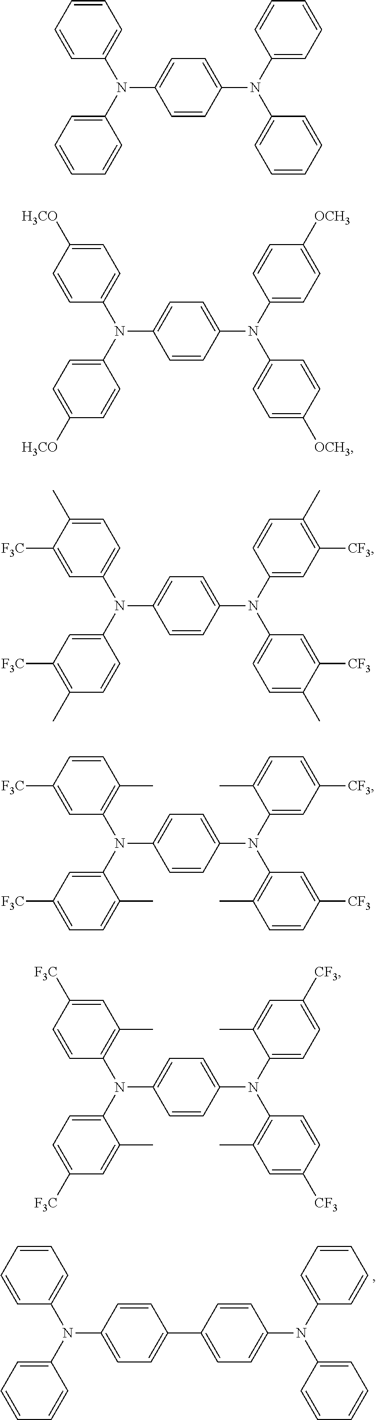

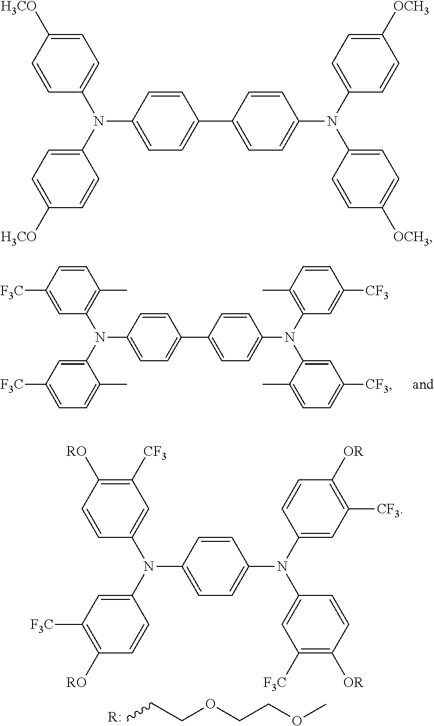

14. The compound of claim 1, wherein the compound is

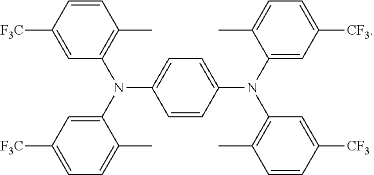 [SMILES: Cc1ccc(C(F)(F)F)cc1N(c1ccc(N(c2cc(C(F)(F)F)ccc2C)c2cc(C(F)(F)F)ccc2C)cc1)c1cc(C(F)(F)F)ccc1C]

15. An electrolyte solution comprising the compound of claim 1 and a hexafluorophosphate salt.

16. A redox-flow battery comprising the electrolyte solution of claim 15.

17. A redox flow battery comprising the compound of claim 1.

18. A method of providing electrical energy comprising flow of a cathoylte and an anolyte, wherein the catholyte comprises the compound of claim 1.

19. The method of claim 17, wherein the catholyte further comprises a hexafluorophosphate salt.
